(19) United States
(12) Patent Application Publication
(10) Pub. No.: US 2023/0012420 A1
(43) Pub. Date: Jan. 12, 2023

(54) METHODS AND COMPOSTIONS FOR INHIBITING P97

(71) Applicant: California Institute of Technology, Pasadena, CA (US)

(72) Inventors: Tsui-Fen Chou, San Marino, CA (US); Shan Li, Pasadena, CA (US); Feng Wang, Pasadena, CA (US); Ting-Yu Wang, Pasadena, CA (US)

(21) Appl. No.: 17/849,075

(22) Filed: Jun. 24, 2022

Related U.S. Application Data
(60) Provisional application No. 63/216,134, filed on Jun. 29, 2021.
(60) Provisional application No. 63/335,459, filed on Apr. 27, 2022.

(51) Int. Cl. C12N 15/113; A61K 31/7088; A61K 38/46; A61K 31/519; A61K 31/4439; A61K 31/428; A61K 31/517; A61K 31/4045; A61K 31/69; A61K 31/538; A61K 31/496; C07K 16/40; A61P 25/28; C12Q 1/6883

(52) U.S. Cl. CPC C12N 15/1137; A61K 31/7088; A61K 38/465; A61K 31/519; A61K 31/4439; A61K 31/428; A61K 31/517; A61K 31/4045; A61K 31/69; A61K 31/538; A61K 31/496; C07K 16/40; A61P 25/28; C12Q 1/6883; C12N 2310/531; C12N 2310/14; C12N 2310/11; C12Q 2600/156; C12Q 2600/158

(57) ABSTRACT

Provided herein are methods and compositions for inhibiting p97, for the treatment of a motor neuron disease in a subject, or a symptom thereof. Upon treatment, the motor neuron disease, or a symptom thereof is reduced in the subject.

### CROSS-REFERENCE TO RELATED APPLICATIONS

The present application claims the benefit of U.S. Provisional Application No. 63/216,134, filed on Jun. 29, 2021, and 63/335,459, filed Apr. 27, 2022, each of which is hereby incorporated by reference in its entirety.

### STATEMENT REGARDING FEDERALLY SPONSORED R&D

This invention was made with government support under Grant No. NS102279 awarded by the National Institutes for Health. The government has certain rights in the invention.

### REFERENCE TO SEQUENCE LISTING

The present application is being filed along with a Sequence Listing in electronic format. The Sequence Listing is provided as file CALTE159_SEQLIST.TXT created and last modified on Jun. 20, 2022, which is 1.3 kB in size. The information in the electronic format of the Sequence Listing is incorporated herein by reference in its entirety.

### FIELD

Some embodiments described herein relate generally to methods related to p97 inhibition for treatment of motor neuron diseases, such as amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, and post-polio syndrome.

### BACKGROUND

Human p97/VCP protein is part of the AAA+ ATPase protein family (Xia, 2016). p97 is ubiquitously expressed and is essential for a variety of cellular activities, including protein homeostasis, mitochondrial quality control, Golgi reassembly and autophagy (Xia, 2016; Chou, 2011). Mutations in p97 cause inclusion body myopathy associated with IBMPFD, (also known as multisystem proteinopathy 1, MSP1; OMIM 167320). To date, more than 40 mutations at 29 different positions in the p97 gene have been found in IBMPFD/ALS (Tang, 2016). The most common mutation in p97 is R155H, and this change accounts for 50% of clinical cases (Mehta, 2013).

Symptoms of IBMPFD include adult-onset muscle weakness (myopathy), early-onset Paget disease of bone (PDB) and premature frontotemporal dementia (FTD) (Al-Obeidi, 2018). One-third of IBMPFD patients will develop FTD (Weihl, 2009). In addition, p97 is associated with 1-2% of cases of family and sporadic ALS, characterized by motor neuron (MN) degeneration (Martin, 2000). Approximately 10% of individuals with IBMPFD have a previous diagnosis of ALS (Johnson, 2010; Kimonis, 2019). Both IBMPFD and ALS lead to motor neuron degeneration (Yi, 2012). p97-mutant neurons develop vacuoles and inclusions, accumulate ubiquitinated proteins, and show aberrant localization of the DNA-binding protein, TDP-43, to the cytosol (Badadani, 2010; Custer, 2010; Nalbandian, 2012; Forman, 2006; Neumann, 2007). A recent study found that the p97 mutation (D395G) impairs disaggregation of PHF-tau, a possible disease-linked mechanism (Darwich, 2020). However, these findings either use mouse models or post-mortem tissue, which reflect some pathological features, but may fail to recapitulate the mechanisms that cause human disease.

Human induced pluripotent stem cells (iPSCs) are a valuable tool for the study of neurological disorders (Marchetto, 2011). Patient-derived iPSCs have been used successfully to model a range of neurological conditions, including ALS, Alzheimer's disease and Parkinson's disease (Sison, 2018; Burkhardt, 2013; Fujimori, 2018; Penney, 2020). Hall et al. modeled p97-related ALS using patient-derived iPSCs, and revealed that cytoplasmic TDP-43, ER stress, mitochondrial function and oxidative stress are key differences between healthy control and p97-mutants MNs (Hall, 2017). While their findings suggest possible disease-linked mechanisms, the global effects of p97 mutants on human MNs remain ambiguous. Since the age of onset and clinical features vary across IBMPFD patients (Al-Obeidi, 2018), genetic background effects should also be taken into account when searching for disruption of cellular functions linked to disease. Moreover, p97-related IBMPFD is thought to occur through gain-of-function. Indeed, disease-associated p97 mutants show enhanced ATPase activity and increased binding to cofactors (Fernandez-Saiz, 2010; Zhang, 2015; Blythe, 2017). In addition, muscle phenotypes in a Drosophila model are rescued by p97 inhibition (Zhang, 2017).

### SUMMARY

In accordance with some embodiments described herein, methods for p97 inhibition for treatment of motor neuron diseases, such as amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, and post-polio syndrome are provided.

Some embodiments provided herein relate to methods of improving, ameliorating or treating a motor neuron disease. In some embodiments, the methods include identifying a subject having a motor neuron disease, or a symptom thereof. In some embodiments, the methods include administering to the subject an effective amount of an agent that promotes inhibition of p97. In some embodiments, the motor neuron disease is reduced after administering the agent that promotes inhibition of p97. In some embodiments, the motor neuron disease is the motor neuron disease is amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, or post-polio syndrome. In some embodiments, the motor neuron disease is caused by at least one mutation in p97. In some embodiments, the mutation in p97 is R155H, D395G, R191Q, or R155C. In some embodiments, the subject having a motor neuron disease expresses one or more genes involved in the RB1/E2F1 pathway differently than in normal subjects. In some embodiments, the genes involved in the RB1/E2F1 pathway comprise E2F1, E2F2, CCNDJ, CDK4, CDK6, DHFR, CDK2, pRB1, RRM2 or TK1. In some embodiments, the agent that promotes inhibition of p97 is an inhibitory nucleic acid molecule. In some embodiments, the inhibitory nucleic acid molecule is an antisense nucleic acid. In some embodiments, the inhibitory nucleic acid molecule is a siRNA. In some embodiments, the inhibitory nucleic acid molecule is a shRNA. In some embodiments, the inhibitory nucleic acid molecule corresponds to or is complementary to at least a fragment of nucleic acid encoding p97. In some embodiments, the agent that promotes inhibition of p97 is a p97 binding antagonist. In some embodiments, the p97 binding antagonist inhibits the binding of p97 to its partners. In some embodiments, the p97 binding antagonist is an antibody against p97 or a fragment of p97. In some embodiments, the antibody is a monoclonal, polyclonal or an antibody fragment selected from the group consisting of Fab, Fab′-SH, Fv, scFv, and (Fab′)₂ fragments. In some embodiments, the agent that promotes inhibition of p97 is a genetic tool. In some embodiments, the genetic tool is selected from the group consisting of a CRISPR/Cas9 system, a zinc finger nuclease system, a TALEN system, a homing endonucleases system or a meganuclease system. In some embodiments, the agent that promotes inhibition of p97 is a small molecule inhibitor. In some embodiments, the small molecule inhibitor that promotes inhibition of p97 is CB-5083, NMS-873, NMS-859, DBeQ, MSC1094308, ML240, p97-IN-1, VCP/p97 inhibitor-1, ML241 hydrochloride, or UPCDC-30245.

Some embodiments provided herein relate to methods of improving, ameliorating or treating a motor neuron disease in a subject in need thereof. In some embodiments, the subject suffers from amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, or post-polio syndrome.

Some embodiments provided herein relate to methods of identifying a subject having a motor neuron disease. In some embodiments, the methods include detecting at least one of a presence, genetic change and/or level of p97 or a level of a product of a gene of the subject selected from the group consisting of: Sox1 (or an ortholog thereof), NES (or an ortholog thereof), Isl1 (or an ortholog thereof), p53 (or an ortholog thereof), γ-H2AX (or an ortholog thereof), Tau (or an ortholog thereof), p-Tau (or an ortholog thereof), Mcm6 (or an ortholog thereof), LC3 (or an ortholog thereof), or a combination of two or more of the listed genes. In some embodiments, detecting a presence, a genetic change and/or a level of (a) and/or (b), wherein (a) and/or (b) are expressed differently and/or have a different genetic status in normal and subjects with motor neuron disease.

Some embodiments provided herein relate to methods of improving, ameliorating, or treating a motor neuron disease. In some embodiments, the methods include detecting at least one of a presence, genetic change and/or level of p97. In some embodiments, the genetic status, level, and/or expression of p97 in the subject is compared to the genetic status, level and/or expression of p97 in the normal subject. In some embodiments, the detection of an abnormal genetic status and/or a high level and/or expression of mutant p97 in the subject relative to the normal subject indicates the presence of a motor neuron disease in the subject. In some embodiments, an effective amount of an agent that promotes inhibition of p97 is administered to the subject with an abnormal genetic status and/or a high level and/or expression of mutant p97.

In some embodiments, the methods include identifying a subject with mutant p97 or a subject who would benefit from inhibiting mutant p97. In some embodiments, the methods include administering an effective amount of an agent to inhibit p97. In some embodiments, the subject is in need of p97 inhibition, and following administration of a p97 inhibiting agent, dysregulated RB1/E2F1 pathway is regulated.

Some embodiments provided herein relate to use of effective amounts of an agent that promotes inhibition of p97 for improvement, amelioration, or treatment a motor neuron disease. In some embodiments, the motor neuron disease is amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, or post-polio syndrome. In some embodiments, the agent that promotes inhibition of p97 is an inhibitory nucleic acid molecule, p97 binding antagonist, a genetic tool, and/or a small molecule inhibitor. In some embodiments, the motor neuron disease is caused by at least one mutation in p97. In some embodiments, the mutation in p97 is R155H, D395G, R191Q, or R155C. In some embodiments, the subject having a motor neuron disease expresses one or more genes involved in the RB1/E2F1 pathway differently than in normal subjects. In some embodiments, the genes involved in the RB1/E2F1 pathway comprise E2F1, E2F2, CCNDJ, CDK4, CDK6, DHFR, CDK2, pRB1, RRM2 or TK1.

In some embodiments, the agents that are used to inhibit p97 in a subject that is in need of treatment for motor neuron disease can be nucleic acid molecules, antagonists that binds and inhibits p97, small molecule inhibitors that inhibit p97 or genetic tools.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIGS. 1A-1F depict the generation of induced human motor neurons (ihMNs) from patient-derived fibroblast cells. FIG. 1A shows the verification of induced pluripotent stem cell (iPSC) identity in cells derived from p97^{R155H/+} patient cells. Scale bar indicates 200 μm. FIG. 1B shows the generation of isogenic lines using clustered regularly interspaced short palindromic repeat (CRISPR)/CRISPR associated protein 9 (CRISPR/Cas9) to correct p97^{R155H} mutation. The sequences used to correct the p97^{R155H} mutation are SEQ ID NO: 1 (GTACGTGGTGGC; H115R template) and SEQ ID NO: 2 (GTCCATGGTGGG; p97^{R155H}). The selected iPS clones were confirmed by DNA sequencing. FIG. 1C shows qPCR analysis of the OCT4 mRNA expression in p97^{R155H/+} and isoWT iPSCs. FIG. 1D depicts a schematic showing strategy for promoting motor neuron differentiation. Days post induction (dpi) and days post maturation (dpm). FIG. 1E shows representative images of morphology and CHAT, ISL1 immunofluorescence staining in mature MNs. Scale bar indicates 50 μm. FIG. 1F illustrates the chip used to record neuron activity in Maxwell (left, CHIP) and the representative footprints of spontaneous firing from a single cluster of Motor Neurons (MNs) at 14 dpm. Immature MNs were seeded on chips in the Maxwell plate and incubated under maturation culture conditions. Neuron activity was recorded at 7, 10, and 14 dpm.

FIGS. 2A-2E depict comparison of MN differentiation of p97^{R155H/+} and isoWT cells. FIG. 2A depicts qPCR analysis of PAX6, SOX1 and OLIG2 at different stages of MN induction. PAX6 and SOX1 were detected at 6 dpi (NEP stage) and OLIG2 was detected at 12 dpi (MNP stage). FIG. 2B depicts qPCR analysis of NES at different stages of MN induction. NES was detected at 6, 12 dpi and 14 dpm. FIG. 2C depicts qPCR analysis of ISL1, HB1 and CHAT at different stages of MN induction. ISL1, HB9 and CHAT were detected at 14 dpm (mature MNs). FIG. 2D depicts cell survival curves during MN maturation. Data represent mean±SD, n=3. *: p<0.05, **: p<0.01 according to unpaired t-test. FIG. 2E depicts western blot of D14 and D20 MNs. FC indicates the average fold change (p97^{R155H/+}/isoWT)_{.}

FIGS. 3A-3E illustrate proteomic and transcriptomic analysis on D14 MNs. FIG. 3A depicts a volcano plot displaying the proteomic changes in p97^{R155H/+} MNs, log 2 (Fold Change) indicates the logarithm to the base 2 of fold change, n=3. FIG. 3B depicts western blot showing increases in NES, MCM6, Filamin 1, and HSP47 are consistent with proteomic data. FC indicates the average fold change in (p97^{R155H/+}/isoWT). FIG. 3C shows a functional enrichment analysis on the proteins affected by p97^{R155H/+}. FIG. 3D illustrates a heatmap displaying the scaled abundance of DEPs related to cell cycle. FIG. 3E illustrates a volcano plot displaying the transcriptomic changes in p97^{R155H/+} MNs.

FIGS. 4A-4G show that p97^{R155H/+} activates the RB1/E2F1 pathway in mature MNs. FIG. 4A depicts RNA-seq data revealing the upregulation of E2F1 and E1F2 genes involved in E2F1 pathway. FIG. 4B depicts RNA-seq data revealing the upregulation of CCNA2, CCNB1, CCNB2, CCND1, CDK4 and CDK6 genes involved in E2F1 pathway. FIG. 4C depicts RNA-seq data revealing the upregulation of DHFR, TK1, CDK2 and RRM2 genes involved in E2F1 pathway. Log 2FC represents log 2 (Fold Change). FIG. 4D illustrates western blot showing the upregulation of E2F1 pathway in p97^{R155H/+} MNs. FC indicates the average fold change of (p97^{R155H/+}/isoWT). FIG. 4E illustrates cell survival results of p97^{R155H/+} MNs treated with DMSO, 200 nM or 400 nM abemaciclib. FIG. 4F illustrates cell survival results of iso p97^{R155H/+} MNs treated with DMSO, 200 nM or 400 nM Abemaciclib. FIG. 4G illustrates Western blot of D14 MNs derived from an unaffected individual carrying WT p97 (WT) and its isogeneic MNs carrying p99^{R155H/+} mutation (isop97^{R155H/+}).

FIGS. 5A-5G show rescue of neuron loss and proteomic changes by p97 inhibitors in p97^{R155H/+} MNs. FIG. 5A depicts neuron loss tested using live cell staining and western blots of cells treated with 400 nM of CB-5083 or 400 nM of NMS-873 for 6 days in p97^{R155H/+} MNs at 20 dpm (A, N=3, *: p<0.05, ** p<0.001 according to unpaired t-test). FIG. 5B depicts neuron loss tested using live cell staining of cells treated with 100 nM of CB-5083 or 100 Nm of NMS-873 for 6 days in isop97^{R155H/+} MNs. *: p<0.05 according to unpaired t-test. FIG. 5C depicts reversal of dysregulated protein levels in p97^{R155H/+} MNs at 20 dpm cells treated with 400 nM of CB-5083 or 400 nM of NMS-873 for 6 days. FC indicates the average fold change. FIG. 5D illustrates PCA of proteomics showing the separation between isoWT MNs treated with DMSO and p97^{R155H/+} MNs treated with DMSO or p97 inhibitors. FIG. 5E depicts a Venn diagram showing that p97 inhibitor treatments prevent changes in proteins which were elevated (up) or reduced (down) in D20 p97^{R155H/+} MNs. Each color indicates the number of DEPs identified from p97^{R155H/+} vs isoWT (yellow), or p97^{R155H/+} MNs treated with DMSO vs CB-5083 (blue) or NMS-873 (red). FIG. 5F represents a heatmap showing that DEPs with 1 Log 2FC1>1 were not seen upon treatment with p97 inhibitors in D20 p97^{R155H/+} MNs. FIG. 5G illustrates a functional enrichment analysis on DEPs identified from D20 p97^{R155H/+} MNS (1), and the DEPs which are reversed (2) or not reversed (3) by p97 inhibitors.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Without being limited by any theory, it is contemplated that mutations in p97 cause inclusion body myopathy associated with Paget disease of bone and frontotemporal dementia (IBMPFD), a rare multisystem degenerative human disorder that afflicts skeletal muscle, bone and brain (Kimonis, 2008; Kimonis, 2008). p97^{R155H/+} is the most frequently identified p97 disease mutant in IBMPFD patients (Kimonis, 2008). One-third of IBMPFD patients will develop premature frontotemporal dementia (FTD) and approximately 10% display features of amyotrophic lateral sclerosis (ALS) (Mehta, 2013; Kimonis, 2019; Kimonis, 2008). Currently, there is no cure for IBMPFD patients.

A variety of cellular functions are disrupted in the presence of mutant p97 (Fernandez-Saiz, 2010; Ludtmann, 2017). For example, increased ATPase activity and altered cofactor binding are seen for p97^{R155H/+} and may underlie IBMPFD pathogenesis (Zhang, 2015; Blythe, 2017). Alterations in the RB1/E2F1 pathway have been observed in ALS neurons in vivo and are thought to contribute to neuron cell death in this disorder (Ranganathan, 2003; Ranganathan, 2010). In addition, ATPase activity of p97 is essential in maintaining CCND1, a critical regulator of the RB1/E2F1 pathway, and inhibition of p97 promotes the degradation of CCND1 (Parisi, 2018). p97 inhibitors can relieve phenotypes, including mitochondrial defects, caused by p97 mutants in adult Drosophila muscle (Zhang, 2017). However, whether p97 inhibitors can prevent neurodegeneration of IBMPFD patient cells is unclear. In addition, the molecular mechanisms underlying neurodegeneration observed in p97 disease mutants remain unknown.

### Definitions

Unless defined otherwise, technical, and scientific terms used herein have the same meaning as commonly understood when read in light of the instant disclosure by one of ordinary skill in the art to which the present disclosure belongs. For purposes of the present disclosure, the following terms are explained below.

The embodiments herein are generally disclosed using affirmative language to describe the numerous embodiments. Embodiments also include ones in which subject matter is excluded, in full or in part, such as substances or materials, method steps and conditions, protocols, or procedures.

In some embodiments, the numbers expressing quantities of ingredients, properties such as concentration, reaction conditions, and so forth, used to describe and claim certain embodiments described herein are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in some embodiments may contain certain errors necessarily resulting from the standard deviation found in their respective testing measurements.

In some embodiments, the terms “a” and “an” and “the” and similar references used in the context of describing a particular embodiment described herein (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate embodiments and does not pose a limitation on the scope of the embodiments otherwise claimed. No language in the specification should be construed as indicating any non-claimed element essential to the practice of any of the embodiments described herein.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The term “inhibit” as used herein has its plain and ordinary meaning as understood in light of the specification and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. The term inhibit may not necessarily indicate a 100% inhibition. A partial inhibition may be realized.

The term “treatment” or “treating” means any administration of a compound or an agent according to the present disclosure to a subject having or susceptible to a condition or disease disclosed herein for the purpose of: 1) preventing or protecting against the disease or condition, that is, causing the clinical symptoms not to develop; 2) inhibiting the disease or condition, that is, arresting or suppressing the development of clinical symptoms; or 3) relieving the disease or condition that is causing the regression of clinical symptoms. In some embodiments, the term “treatment” or “treating” refers to relieving the disease or condition or causing the regression of clinical symptoms.

The term “effective amount” is meant as the amount of an agent required to reduce the symptoms of a disease relative to an untreated subject. The effective amount of agent(s) used to practice any of the embodiments described herein for therapeutic treatment of a motor neuron disease varies depending upon the manner of administration, the age, body weight, and general health of the subject. Ultimately, a physician or veterinarian will decide the appropriate amount and dosage regimen. Such amount is referred to as an “effective” amount.

Preferred embodiments are described herein, including the best mode known to the inventors for carrying out certain embodiments. Variations on those preferred embodiments will become apparent to those of ordinary skill in the art upon reading the foregoing description. It is contemplated that skilled artisans can employ such variations as appropriate, and embodiments can be practiced otherwise than specifically described herein. Accordingly, many embodiments include all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

Furthermore, numerous references have been made to patents and printed publications throughout this specification. Each of the above cited references and printed publications are herein individually incorporated by reference in their entirety.

Embodiments disclosed herein are illustrative of the principles of the disclosure. Other modifications that can be employed can be within the scope of the disclosure. Thus, by way of example, but not of limitation, alternative configurations can be utilized in accordance with the teachings herein. Accordingly, embodiments are not limited to that precisely as shown and described.

### Inhibition of Mutant p97

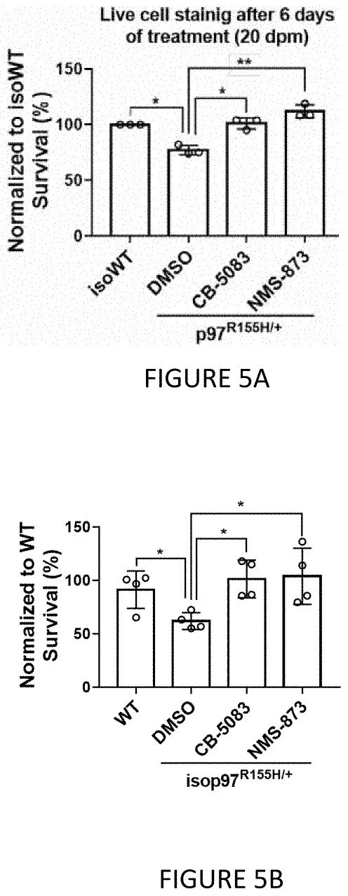

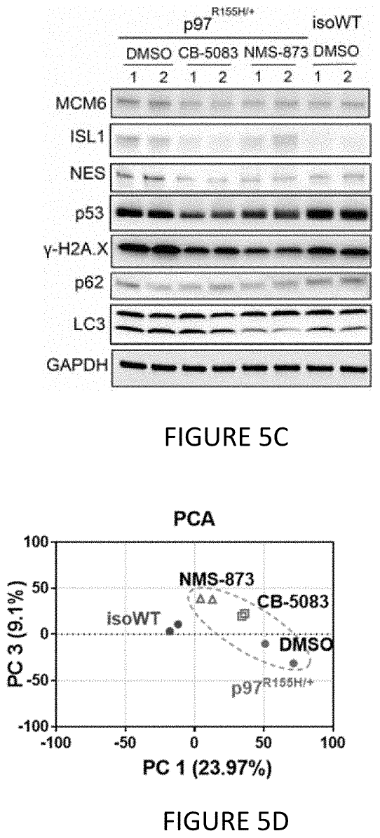

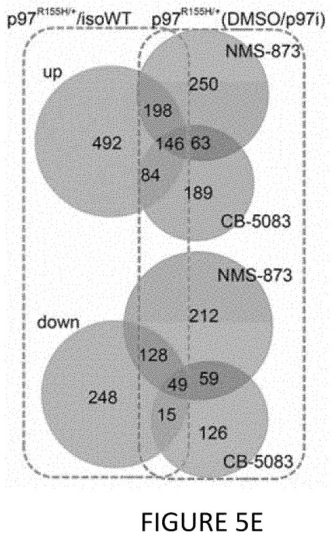

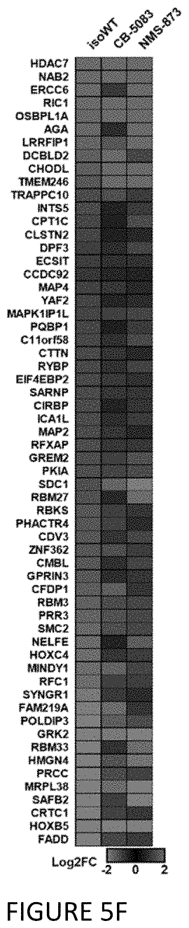

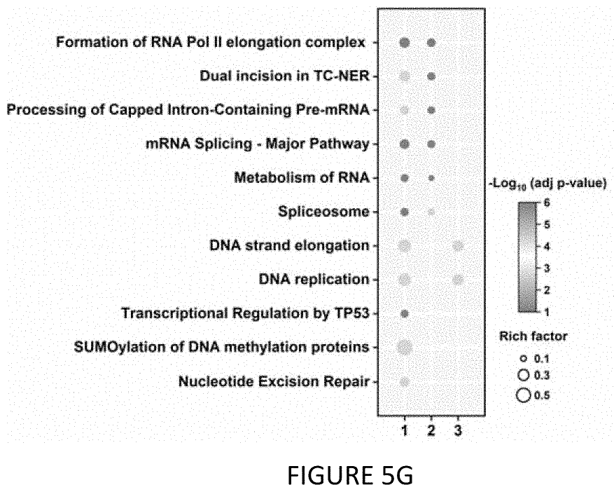

Inhibition of mutant p97 has been observed to rescue the neurodegeneration observed in motor neuron IBMPFD whereas treatment with vehicle has no effect (see FIGS. 5A-5D). Accordingly, in some embodiments described herein, methods of treatment for motor neuron diseases are provided. The methods can comprise administering an effective amount of an agent that promotes inhibition of p97 in the subject with a motor neuron disease. Following administration of an agent that promotes inhibition of p97, the motor neuron disease or a symptom thereof is reduced.

Without being limited by any theory, more than 40 mutations at 29 different positions in the p97 gene have been found in IBMPFD/ALS (Tang, 2016). In some embodiments, mutant p97 that is inhibited is R155H. In some embodiments, mutant p97 that is inhibited is D395G. In some embodiments, mutant p97 that is inhibited is R191Q. In some embodiments, mutant p97 that is inhibited is R155C.

Various agents can be used to inhibit p97 in a subject that is in need of treatment for motor neuron disease. For example, a nucleic acid molecule can be used to inhibit p97. In some embodiments, an antagonist that binds and inhibits p97 can be used. As another example, small molecule inhibitors that inhibit p97 can be used. As still yet another example, a genetic tool can be used to inhibit p97.

In some embodiments, inhibition of p97 reduces a motor neuron disease or a symptom thereof, wherein the motor neuron disease is amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, or post-polio syndrome. In some embodiments, the methods include administering a therapeutically effective amount of an agent that promotes inhibition of p97 to a subject in need thereof.

In some embodiments, the subject has ALS. In some embodiments, the method further comprises determining whether the subject has ALS, and the effective amount of p97 inhibiting agent is administered if the subject has ALS. In some embodiments, the subject has IBMPFD. In some embodiments, the method further comprises determining whether the subject has IBMPFD, and the effective amount of p97 inhibiting agent is administered if the subject has IBMPFD. In some embodiments, the subject has progressive bulbar palsy. In some embodiments, the method further comprises determining whether the subject has progressive bulbar palsy, and the effective amount of p97 inhibiting agent is administered if the subject has progressive bulbar palsy. In some embodiments, the subject has primary lateral sclerosis. In some embodiments, the method further comprises determining whether the subject has primary lateral sclerosis, and the effective amount of p97 inhibiting agent is administered if the subject has primary lateral sclerosis. In some embodiments, the subject has progressive muscular atrophy. In some embodiments, the method further comprises determining whether the subject has progressive muscular atrophy, and the effective amount of p97 inhibiting agent is administered if the subject has progressive muscular atrophy. In some embodiments, the subject has spinal muscular atrophy. In some embodiments, the method further comprises determining whether the subject has spinal muscular atrophy, and the effective amount of p97 inhibiting agent is administered if the subject has spinal muscular atrophy. In some embodiments, the subject has Kennedy's disease. In some embodiments, the method further comprises determining whether the subject has Kennedy's disease, and the effective amount of p97 inhibiting agent is administered if the subject has Kennedy's disease. In some embodiments, the subject has post-polio syndrome. In some embodiments, the method further comprises determining whether the subject has post-polio syndrome, and the effective amount of p97 inhibiting agent is administered if the subject has post-polio syndrome.

In accordance with any of the embodiments described above, an effective amount of a nucleic acid molecule that corresponds to or is complementary to at least a fragment of nucleic acid encoding p97 is administered to inhibit p97. In accordance with any of the embodiments described above, the nucleic acid molecule is a siRNA. In some embodiments, the nucleic acid molecule is a shRNA. In accordance with any of the embodiments described above, the nucleic acid molecule is an antisense nucleic acid.

In accordance with any of the embodiments described above, an effective amount of antagonist that binds and inhibits p97 is administered. In accordance with any of the embodiments described above, the antagonist is an antibody against p97 or a fragment of p97. In accordance with any of the embodiments described above, the antibody is a monoclonal, polyclonal or an antibody fragment selected from the group consisting of Fab, Fab′-SH, Fv, scFv, and (Fab′)2 fragments.

In accordance with any of the embodiments described above, a genetic tool is administered to inhibit p97. In accordance with any of the embodiments described above, the genetic tool to inhibit p97 is a CRISPR/Cas9 system. In accordance with any of the embodiments described above, the genetic tool to inhibit p97 is a zinc finger nuclease system. In accordance with any of the embodiments described above, the genetic tool to inhibit p97 is a TALEN system. In accordance with any of the embodiments described above, the genetic tool to inhibit p97 is a homing endonucleases system. In accordance with any of the embodiments described above, the genetic tool to inhibit p97 is a meganuclease system.

In accordance with any of the embodiments described above, a small molecule inhibitor is administered to inhibit p97. In accordance with any of the embodiments described above, the small molecule inhibitor to inhibit p97 is CB-5083. As used herein, the term CB-5083 has its ordinary meaning as understood in light of the specification and refers to a p97 AAA ATPase/VCP inhibitor that is orally bioavailable, and that selectively inhibits p97, and that has the chemical formula C₂₄H₂₃N₅O₂, with the chemical name of 1-(4-(benzylamino)-7,8-dihydro-5H-pyrano[4,3-d]pyrimidin-2-yl-2-methyl-1H-indole-4-carboxamide, and which has the chemical structure:

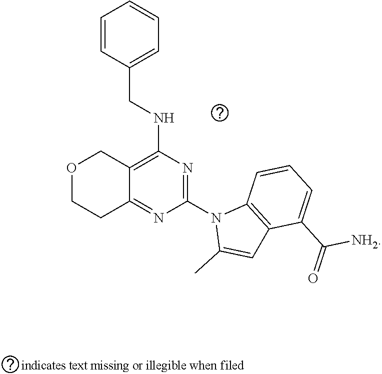 [SMILES: Cc1cc2c(C(N)=O)cccc2n1-c1nc2c(c(NCc3ccccc3)n1)COCC2]

In some embodiments, the small molecule inhibitor of p97 is CB-5083 or any functional salt, derivative, or analogue thereof.

In accordance with any of the embodiments described above, the small molecule inhibitor to inhibit p97 is NMS-873. As used herein, the term NMS-873 has its ordinary meaning as understood in light of the specification and refers to an allosteric p97 AAA ATPase/VCP inhibitor that is orally bioavailable, and that selectively inhibits p97, and that has the chemical formula C₂₇H₂₈N₄O₃S₂, with the chemical name of 3-[3-cyclopentylsulfanyl-5-[[3-methyl-4-(4-methylsulfonylphenyl)phenoxy]methyl]-1,2,4-triazol-4-yl]pyridine, and which has the chemical structure:

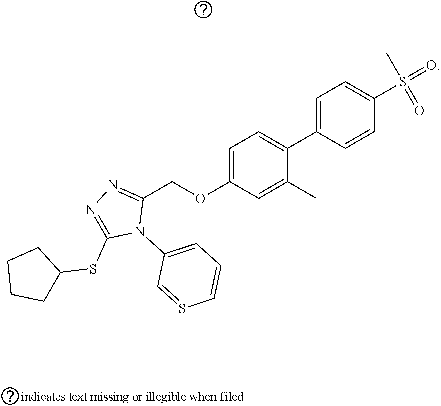

In some embodiments, the small molecule inhibitor of p97 is NMS-873 or any functional salt, derivative, or analogue thereof.

In accordance with any of the embodiments described above, the small molecule inhibitor to inhibit p97 is NMS-859. As used herein, the term NMS-859 has its ordinary meaning as understood in light of the specification and refers to a small molecule p97 AAA ATPase/VCP inhibitor, and that selectively inhibits p97, and that has the chemical formula C₁₅H₁₂ClN₃O₃S, with the chemical name of 2-chloro-N-(3-((1,1-dioxidobenzo[d]isothiazol-3-yl)amino)phenyl)acetamide, and which has the chemical structure:

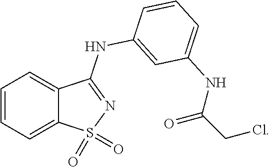 [SMILES: O=C(CCl)Nc1cccc(NC2=NS(=O)(=O)c3ccccc32)c1]

In some embodiments, the small molecule inhibitor of p97 is NMS-859 or any functional salt, derivative, or analogue thereof.

In accordance with any of the embodiments described above, the small molecule inhibitor to inhibit p97 is DBeQ. As used herein, the term DBeQ has its ordinary meaning as understood in light of the specification and refers to an ATP-competitive p97/VCP inhibitor, and that inhibits p97, and that has the chemical formula C₂₂H₂₀N₄, with the chemical name of N²,N⁴-Bis(phenylmethyl)-2,4-quinazolinediamine, and which has the chemical structure:

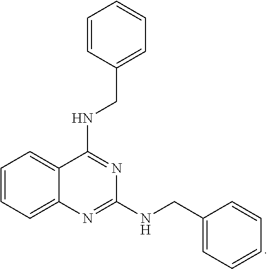 [SMILES: c1ccc(CNc2nc(NCc3ccccc3)c3ccccc3n2)cc1]

In some embodiments, the small molecule inhibitor of p97 is DBeQ or any functional salt, derivative, or analogue thereof.

In accordance with any of the embodiments described above, the small molecule inhibitor to inhibit p97 is MSC1094308. As used herein, the term MSC1094308 has its ordinary meaning as understood in light of the specification and refers to an allosteric p97 AAA ATPase/VCP inhibitor, and that inhibits p97, and that has the chemical formula C₂₉H₂₉F₃N₄, with the chemical name of N-((6-fluoro-2,3,4,9-tetrahydro-1H-carbazol-3-yl)methyl)-4,4-bis(4-fluorophenyl)butan-1-amine, and which has the chemical structure:

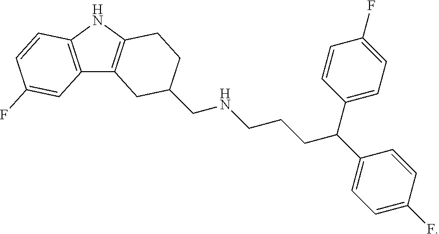 [SMILES: Fc1ccc(C(CCCNCC2CCc3[nH]c4ccc(F)cc4c3C2)c2ccc(F)cc2)cc1]

In some embodiments, the small molecule inhibitor of p97 is MSC1094308 or any functional salt, derivative, or analogue thereof.

In accordance with any of the embodiments described above, the small molecule inhibitor to inhibit p97 is ML240. As used herein, the term ML240 has its ordinary meaning as understood in light of the specification and refers to a p97 AAA ATPase/VCP inhibitor, and that selectively inhibits p97, and that has the chemical formula C₂₃H₂₀N₆O, with the chemical name of 2-(2-Amino-1H-benzimidazole-1-yl)-8-methoxy-N-phenylmethyl)-4-quinazolinamine, and which has the chemical structure:

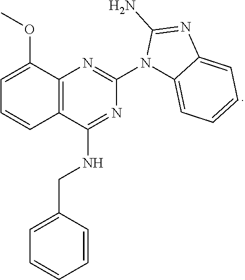 [SMILES: COc1cccc2c(NCc3ccccc3)nc(-n3c(N)nc4ccccc43)nc12]

In some embodiments, the small molecule inhibitor of p97 is ML240 or any functional salt, derivative, or analogue thereof.

In accordance with any of the embodiments described above, the small molecule inhibitor to inhibit p97 is p97-IN-1. As used herein, the term p97-IN-1 has its ordinary meaning as understood in light of the specification and refers to a p97/VCP inhibitor, and that selectively inhibits p97, and that has the chemical formula C₂₄H₂₄N₆O, and which has the chemical structure:

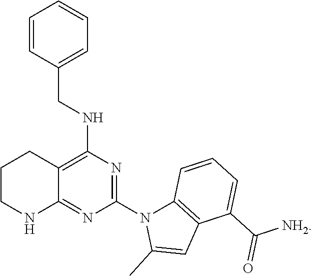 [SMILES: Cc1cc2c(C(N)=O)cccc2n1-c1nc2c(c(NCc3ccccc3)n1)CCCN2]

In some embodiments, the small molecule inhibitor of p97 is p97-IN-1 or any functional salt, derivative, or analogue thereof.

In accordance with any of the embodiments described above, the small molecule inhibitor to inhibit p97 is VCP/p97 inhibitor-1. As used herein, the term VCP/p97 inhibitor-1 has its ordinary meaning as understood in light of the specification and refers to a p97/VCP inhibitor, and that selectively inhibits p97, and that has the chemical formula C₂₄H₂₆BN₅O₄S, and which has the chemical structure:

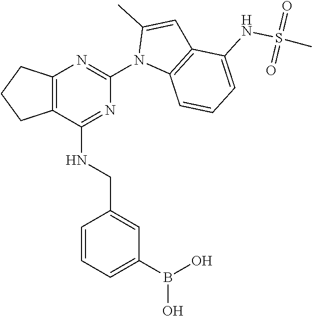 [SMILES: Cc1cc2c(NS(C)(=O)=O)cccc2n1-c1nc2c(c(NCc3cccc(B(O)O)c3)n1)CCC2]

In some embodiments, the small molecule inhibitor of p97 is VCP/p97 inhibitor-1 or any functional salt, derivative, or analogue thereof.

In accordance with any of the embodiments described above, the small molecule inhibitor to inhibit p97 is ML241 hydrochloride. As used herein, the term ML241 hydrochloride has its ordinary meaning as understood in light of the specification and refers to a p97 AAA ATPase/VCP inhibitor, and that selectively inhibits p97, and that has the chemical formula C₂₃H₂₅ClN₄O, with the chemical name of 2-(2H-benzo[b][1,4]oxazin-4(3H)-yl)-N-benzyl-5,6,7,8-tetrahydroquinazolin-4-amine hydrochloride, and which has the chemical structure:

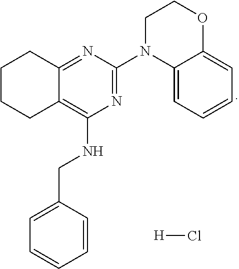 [SMILES: c1ccc(CNc2nc(N3CCOc4ccccc43)nc3c2CCCC3)cc1 ; Cl]

In some embodiments, the small molecule inhibitor of p97 is ML241 hydrochloride or any functional salt, derivative, or analogue thereof.

In accordance with any of the embodiments described above, the small molecule inhibitor to inhibit p97 is UPCDC-30245. As used herein, the term UPCDC-30245 has its ordinary meaning as understood in light of the specification and refers to an allosteric p97 AAA ATPase/VCP inhibitor, and that selectively inhibits p97, and that has the chemical formula C₂₈H₃₈FN₅, with the chemical name of 1-(3-(5-Fluoro-1H-indol-2-yl)phenyl)-N-(2-(4-isopropylpiperazin-1-yl)ethyl)piperidin-4-amine, and which has the chemical structure:

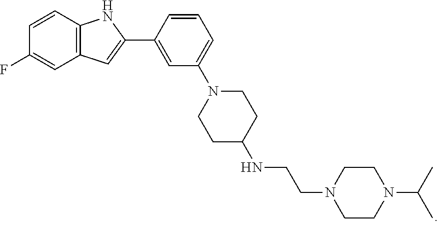 [SMILES: CC(C)N1CCN(CCNC2CCN(c3cccc(-c4cc5cc(F)ccc5[nH]4)c3)CC2)CC1]

In some embodiments, the small molecule inhibitor of p97 is UPCDC-30245 or any functional salt, derivative, or analogue thereof.

In some embodiments, inhibition of p97 increases or decreases the level or expression of genes associated with RB1/E2F1 pathway. Examples of genes associated with RB1/E2F1 pathway that are increased or decreased by inhibition of p97 include, but not limited to, E2F1, E2F2, CCND1, CDK4, CDK6, DHFR, TK1, RRM2, CDK2, CCNA2, CCNB1, and CCNB2.

In some embodiments, inhibition of p97 increases or decreases the level or expression of genes associated with progression of motor neuron disease. Examples of genes associated with progression of neuro degeneration that are increased or decreased by inhibition of p97 include, but not limited to, SOX1, NES, ISL1, and OLIG2. In some embodiments, inhibition of p97 increases or decreases the level or expression of biomarkers of neurodegeneration. Examples of biomarkers of neurodegeneration that are increased or decreased by depletion of p97 include, but not limited to, LC3, p53, γ-H2AX, Tau, p-Tau, Nestin, Filamin 1, MCM6, and HSP47.

As described herein, inhibiting p97 can treat, inhibit, or ameliorate motor neuron disease symptoms. As disclosed herein, amelioration is used in a broad sense to refer to at least a reduction in the magnitude of a parameter, e.g., symptom, associated with the pathological condition being treated. In some embodiments, the method can completely inhibit, e.g., prevented from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

### Dosage and Administration of p97 Inhibitors for Treating Motor Neuron Disease

Doses of p97 inhibitors can be readily determined for a given subject based on their body mass, disease type and state, and desired aggressiveness of treatment. In some embodiments, inhibitors of p97 are administered at a dose ranging from 1 mg/kg to 200 mg/kg, such as 1, 2, 3, 4, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 mg/kg, or an amount within a range defined by any two of the aforementioned values. The composition may be administered twice daily, once daily, twice weekly, once weekly, or once monthly, or at a frequency within a range defined by any two of the aforementioned values.

In accordance with embodiments described herein, inhibitors of p97 can be administered by any suitable route of administration. Without limitation, the inhibitors of p97 can be administered to the subject via oral administration, rectum administration, transdermal administration, intranasal administration, or inhalation. In some embodiments, the inhibitors of p97 are administered to the subject orally. In some embodiments, the inhibitors of p97 can be administered by injection or in the form of a tablet, capsule, patch or a drink.

Pharmaceutically acceptable carriers are ones which are nontoxic to the cell or mammal being exposed thereto at the dosages and concentrations employed. Pharmaceutically acceptable carriers in accordance with methods and uses and compositions herein can comprise, but not limited to, organic or inorganic, solid or liquid excipients which is suitable for the selected mode of application such as oral application or injection and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as and nonionic surfactants such as TWEEN™ surfactant, polyethylene glycol (PEG), and PLURONICS™ surfactant. Auxiliary, stabilizer, emulsifier, lubricant, binder, pH adjustor controller, isotonic agent and other conventional additives may also be added to the carriers.

### Methods of Treating a Motor Neuron Disease

Described herein are methods of treatment of a motor neuron disease. In some embodiments, the presence of mutant p97 in a subject in need of treatment for motor neuron disease is determined. In some embodiments, provided are methods for treating a motor neuron disease in a subject that is amenable to treatment by inhibiting p97.

Various methods can be used to inhibit mutant p97 in a subject and reduce the motor neuron disease, or a symptom thereof. For example, an ATP-competitor can be used to inhibit the enzyme activity of p97. In some embodiments, treatment with an allosteric p97 inhibitor can be used to inhibit p97.

In some embodiments, provided are methods for treating a motor neuron disease. In some embodiments, the motor neuron disease is amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, or post-polio syndrome. In some embodiments, the methods include administering a therapeutically effective amount of an agent that promotes inhibition of p97, to a subject in need thereof.

In various embodiments, the method is for treating ALS, including inhibition of p97 in a subject who is in need of treatment for ALS, thereby treating the subject. In various embodiments, the method is for treating IBMPFD, including inhibition of p97 in a subject who is in need of treatment for IBMPFD, thereby treating the subject. In various embodiments, the method is for treating progressive bulbar palsy, including inhibition of p97 in a subject who is in need of treatment for progressive bulbar palsy, thereby treating the subject. In various embodiments, the method is for treating primary lateral sclerosis, including inhibition of p97 in a subject who is in need of treatment for primary lateral sclerosis, thereby treating the subject. In various embodiments, the method is for treating progressive muscular atrophy, including inhibition of p97 in a subject who is in need of treatment for progressive muscular atrophy, thereby treating the subject. In various embodiments, the method is for treating spinal muscular atrophy, including inhibition of p97 in a subject who is in need of treatment for spinal muscular atrophy, thereby treating the subject. In various embodiments, the method is for treating Kennedy's disease, including inhibition of p97 in a subject who is in need of treatment for Kennedy's disease, thereby treating the subject. In various embodiments, the method is for treating post-polio syndrome, including inhibition of p97 in a subject who is in need of treatment for post-polio syndrome, thereby treating the subject.

In some embodiments as described above, the methods further comprise identifying a subject with mutant p97 or a subject who would benefit from inhibiting mutant p97. The methods can comprise administering an effective amount of an agent to inhibit p97. In some embodiments, the subject is in need of p97 inhibition, and following administration of p97 inhibiting agent, the motor neuron disease, or a symptom thereof is reduced in the subject.

In some embodiments, the methods further comprise identifying a subject with mutant p97 or a subject who would benefit from inhibiting mutant p97. The methods can comprise administering an effective amount of an agent to inhibit p97. In some embodiments, the subject is in need of p97 inhibition, and following administration of a p97 inhibiting agent, dysregulated RB1/E2F1 pathway is regulated.

In some embodiments, the methods further comprise identifying a subject with mutant p97 or a subject who would benefit from inhibiting mutant p97. The methods can comprise administering an effective amount of an agent to inhibit p97. In some embodiments, the subject is in need of p97 inhibition, and following administration of a p97 inhibiting agent, the level or expression of genes associated with progression of motor neuron disease are regulated. Examples of genes associated with progression of neuro degeneration that are increased or decreased by inhibition of p97 include, but not limited to, SOX1, NES, ISL1, and OLIG2. In some embodiments, inhibition of p97 increases or decreases the level or expression of biomarkers of neurodegeneration. Examples of biomarkers of neurodegeneration that are increased or decreased by inhibition of p97 include, but not limited to, LC3, p53, γ-H2AX, Tau, p-Tau, Nestin, Filamin 1, MCM6, and HSP47.

In some embodiments, the p97 inhibiting agent is administered to the subject until a motor neuron disease, or a symptom thereof in the subject is reduced. Optionally, the p97 inhibiting agent is administered to the subject after a motor neuron disease, or a symptom thereof in the subject is reduced, for example to solidify or maintain the subject free of a motor neuron disease.

As described herein, inhibiting mutant p97 can treat, inhibit, or ameliorate motor neuron disease, or a symptom thereof. As disclosed herein, amelioration is used in a broad sense to refer to at least a reduction in the magnitude of a parameter, e.g., symptom, associated with the pathological condition being treated. In some embodiments, the method can completely inhibit, e.g., prevented from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

### EXAMPLES

Some aspects of the embodiments discussed above are disclosed in further detail in the following examples, which are not in any way intended to limit the scope of the present disclosure.

### Experimental Material and Methods

The following experimental methods were used for Examples 1-6 described below.

iPSCs Reprogramming and Maintenance

Human fibroblast from patient with p97^{R155H/+} mutant was obtained from the Coriell Institute (Coriell code: GM21851) and reprogrammed into iPSCs as a published protocol using episomal vectors (Okita, 2013). Briefly, five non-integrating episomal vectors pCE-hOCT3/4, pCE-hSK, pCE-hUL, pCE-mp53DD, and pCXB-EBNA1 purchased from Addgene were introduced into fibroblasts using the Human Dermal Fibroblast Nucleofector Kit (Lonza, cat #VPD-1001) and Nucleofector 2b Device (Lonza, cat #AAB-1001, program U-023) according to the manufacturer's protocol. The cells were then cultured on Matrigel (BD Biosciences, cat #354230) coated 6-well plates in DMEM (containing 15% FBS and 10 ng/mL FGF) for 2 days and in E7 medium for another 8 days. Cells were cultured in mTeSR1 medium (STEMCELL, cat #85850) from day 10 until iPSC colonies appeared. Colonies were picked and cultured in mTesR1 with 10 nM/mL rock inhibitor (Sigma, cat #Y0503) on Matrigel-coated plates. After 24 hours, the rock inhibitor was removed, and the medium was changed every day. The iPSCs were passaged using ReLeSR (STEMCELL, cat #85872) every 5 to 7 days at split ratios of 1:6 to 1:9 when they reached ˜80% confluence. Cells were tested for mycoplasma routinely using MycoAlert Mycoplasma Detection Kit (Lonza, cat #LT07-118). Quantitative RT-PCR (qPCR) method followed manufacturer instructions for the hPSC Genetic Analysis Kit (Stemcell, cat #07550) to detect recurrent karyotypic abnormalities reported in iPSCs. Clones that passed the RT-PCR assay were sent for karyotyping (ThermoFisher, KaryoStat™ assay) after 10 passages. CRISPR/cas9-mediated genome editing of iPSCs.

Guide RNA, Alt-R® CRISPR-Cas9 tracrRNA ATTO™ 550 (IDT, cat #1075928), and Alt-R® S.p. HiFi Cas9 Nuclease V3 (IDT, cat #1081061) were used to form Ribonucleoprotein (RNP) complexes following the manufacturer's protocol. The H155R single strand donor DNA sequence (H155R-Reverse Complement) contains a T to C correction at the R155H mutation site of the human p97 gene. As an example, the guide RNA sequence is 5′-CCACAGCACGCATCCCACCA-3′ (SEQ ID NO: 3) and the H155R reverse complement sequence is 5′-ATCTGTTTCCACCACTTTGAACTCCACAGCACGCATG CCACCACGTACAAGAAAAATGTCTCCTGCGAGAGCA AACAGTA-3′ (SEQ ID NO: 4). The donor sequence includes a silent G to T mutation at the PAM site to avoid re-cutting by Cas9. Another silent mutation, C to G, brings in the SphI digestion site to identify edited clones. The iPSC medium was changed for IPSCs (50%-80% confluence) with fresh mTeSR1 containing 10 rock inhibitor and 5 μM L755507 (Sigma, cat #SML1362) one day before electroporation. RNP complexes and the single-strand donor DNA were transfected into the iPSCs using the Human Stem Cell Nucleofector Starter Kit (Lonza, cat #VPH-5002) and Nucleofector 2b Device (Lonza, cat #AAB-1001, program B-016) according to manufacturer instructions. Cells were plated on Matrigel-coated 6-well plates in mTesR1 medium containing clone R supplement (STEMCELL, cat #05888) at low density. After 24 hours, the cells were observed with a fluorescence microscope to confirm the RNP protein was transfected into the cells. Clone R was removed after 48 hours. Colonies were picked 7-10 days after transfection, and cultured on Matrigel-coated 24-well plate in mTesR1 medium containing 10 μM rock inhibitor for another 7-10 days. Then individual colonies were manually split to 2 halves. One half was used for genomic DNA extraction with QuickExtract™ DNA Extraction Solution (Epibio, cat #BQ0901S). The other half was maintained in 24-well plates. PCR reactions were performed to amplify regions covering the R155H mutation site using VCP-Ex5 (F+R) primers (VCP-Ex5-F: 5′-TGGAGTTGGGGAGAGGTAGGG-3′ (SEQ ID NO: 5); VCP-Ex5-R 5′-AAAATCGGATACTGGAATCAGGGAGA-3′(SEQ ID NO: 6)) and Platinum Taq DNA polymerase (Invitrogen, cat #10966018) following the user guide. The PCR products were digested using the SphI restriction enzyme (NEB, cat #R3182). Clones that could be digested by SphI were further sequenced by Laragen DNA Sequencing Service.

### Motor Neuron Differentiation and Maturation

Human iPSCs were differentiated into MNs using a previously published protocol (Du, 2015). A basal induction medium which contained advanced DMEM/F12 (Gibco, cat #12634-010) and neurobasal medium (Gibco, cat #21103-049) (1:1 v/v), 1% 50× B27 (Gibco, cat #17504-044), 0.5% 100×N2(Gibco, cat #17502-048), 0.1 mM Ascorbic Acid (Sigma, cat #A4544), 1% 100× Glutamax (Gibco, cat #35050-061), and 1% 100× Antibiotic-Antimycotic (Gibco, cat #15240-062) was prepared. When iPSCs reached ˜80% confluence, they were in basal induction medium containing 3 μM CHIR99021 (Cayman Chemical, cat #13122), 2 μM SB431542 (Cayman Chemical, cat #13031) and 2 μM DMH-1 (Tocris, cat #4126), plated on Geltrex (Gibco, cat #A1413201) coated 6-well plates and cultured for 6 days to generate NSC cells. The culture medium was changed every other day. The NSC cells were then dissociated with accutase (STEMCELL, cat #07920) and further induced by culturing with the induction basal medium containing 1 μM CHIR99021, 2 μM SB431542, 2 μM DMH-1, 0.1 μM RA (Sigma, cat #554720), and 0.5 μM purmorphamine (R&D, cat #4551) to become OLIG2+ motor neuron progenitors (MNPs). The medium was changed every other day. MNPs can be expanded for several passages with the induction basal medium containing 3 μM CHIR99021, 2 μM DMH-1, 2 μM SB431542, 0.1 μM RA, 0.5 μM Purmorphamine, and 0.5 mM VPA (R&D, cat #2815), and split 1:6 every six days with accutase. MNPs were dissociated with accutase, transferred into poly-Hema (Sigma, cat #P3932) coated flasks, and treated with basal induction medium containing 0.5 μM RA and 0.1 μM purmorphamine for another six days on the shaker to let the cells form HB9+ EBs (MN-sus) and expand. Half of the medium was changed every other day. MN-sus can be dissociated into single cells with accutase and can be frozen for future maturation culture.

The maturation of MNs was followed using a 14 day protocol from BrainXell. Briefly, the MNs were thawed and plated on poly-lysine (Sigma, cat #P7886) coated plates in MN maturation medium containing DMEM/F12 (Gibco, cat #11330-032) and neurobasal medium (1:1 v/v), 2% 50× B27, 1% 100× N2, 0.25% 100× Glutamax, 10 ng/mL BDNF (Peprotech, cat #450-02), 10 ng/mL GDNF(Peprotech, cat #450-10), 1 ng/mL TGF-β1 (Peprotech, cat #100-21c), and 1× Brainfast supplement (BrainXell). The medium was changed with the same medium containing 15 μg/mL geltrex on day 1. An equal volume of maturation medium (excluding Geltrex) was added on day 4. Half of the medium (excluding the Brainfast supplement and Geltrex) was changed twice weekly from day 7. The MNs can be maintained for at least three weeks. For the p97 inhibitor treatment assays, medium was changed with fresh medium containing 400 nM of CB-5083 or NMS-873 (purchased from MedKoo; CB-5083, cat #206489; NMS-873, cat #406458), or same volume of DMSO at D14, and then incubated for 6 days. The cells were harvested on D20.

Quantitative Real-Time PCR (qRT-PCR)

Cells were harvested and pellets were resuspended in DPBS/TRIzol-LS mixture (Ambion, cat #10296010; v/v 1:3). Total RNA samples were extracted from the TRIzol-LS mixture using Direct-zol RNA MiniPrep plus kit (Zymo Research, cat #R2072) according to the manufacturer's instructions. The RNA concentration was measured with NanoDrop Lite UV visible spectrophotometer (Thermo Scientific, cat #S/N 2361). 1 μg of total RNA was used to reverse transcribe complementary DNA using the SensiFAST™ cDNA Synthesis Kit (Bioline, cat #BIO-65054). qRT-PCR reactions were performed using SensiFAST Probe HI-ROX Mix (Bioline, cat #BIO-82020) on the QuantStudio™ 5 Real-Time PCR System (Thermo Scientific, cat #A28140). 2\hat{ }(−delta CT) was calculated by normalizing to GAPDH levels. All sample reactions were carried out in triplicates. The error bar reveals the standard deviation of the mean from all of the cell lines used in this paper. The primer probes used in this study are provided in Table 1.

TABLE 1

| Target | Catalog number |
|---|---|
| GAPDH | Hs02786624 g1 |
| OCT4 | Hs00999634gH |
| PAX6 | Hs01088106 g1 |
| NES | Hs04187831 g1 |
| SOX1 | Hs01057642 s1 |
| OLIG2 | Hs00300164 s1 |
| HB9 | Hs00232128 m1 |
| ISL1 | Hs00158126 m1 |
| CHAT | Hs00758143m1 |

### Immunocytochemistry Staining and Imaging

The iPSCs were stained using an Alkaline Phosphatase Staining Kit (StemTAG, cat #CBA-300) and PSC Immunocytochemistry Kit (Invitrogen, cat #A24881) by following the manufacturer's protocol. NSC cells were stained using the Human Neural Stem Cell Immunocytochemistry Kit (Invitrogen, cat #A24354) following the manufacturer's protocol. MNP and MN cells were fixed with 4% PFA at room temperature for 15 min or with cold methanol for 5 min, blocked with 10% donkey serum and 0.1% Triton X-100 in 1×DPBS for 1 hour. The cells were then incubated with the primary antibodies (Table 2) for 2 hours at room temperature and washed 3 times with DPBS containing 0.1% BSA, and incubated with species-specific Alexa Fluor 488-conjugated secondary antibody (donkey anti-mouse immunoglobulin G (IgG), 1:500, Life Technologies) or Alexa Fluor 555-conjugated secondary antibody (Rabbit anti-goat IgG, 1:500, Life Technologies) for 30 min. Cells were then washed 3 times with DPBS and nuclei stained using Hochest (1 Ong/mL) for 10 min. Cell images were acquired using the EVOS FL Auto 2 Imaging System (Invitrogen, cat #AMAFD2000).

### Western Blot

MN cells were scraped off plates, washed with DPBS, and centrifuged at 300 g for 4 min to remove supernatant. Pellets were frozen at −80° C. Pellets were resuspended with 100 lysis buffer (50 mM Tris-HCl pH 8.0, 150 mM NaCl, 1% Triton X-100 with protease inhibitor tablet, 50 μM MG132, and 50 mM NEM), and incubated on ice for 10 min with occasional vortexing. After that, samples were centrifuged at 15000 rpm at 4° C. for 10 min and transferred 90 μL cell lysate into a 1.5 mL tube. Total soluble protein concentrations were measured using the Bradford test (Bio-Rad, cat #5000006). 30 μL 4×Laemmli sample buffer (Bio-Rad, cat #161-0774) was added and samples were boiled for 5 min. An equal amount of protein was loaded and separated using 4-20% Mini-PROTEAN TGX precast gels (Bio-Rad, cat #456-1096) and transferred to nitrocellulose membranes using Trans-Blot Turbo Transfer System (Bio-Rad, cat #170-4155). Membranes were blocked with 1×TBST with 5% w/v nonfat milk, incubated with primary antibodies for 2 h at room temperature or overnight at 4° C., and incubated with HRP-conjugated secondary antibodies (Bio-Rad, 1:3000 dilution) for 2 h at room temperature. Then ECL reagent (MilliporeSigma, cat #WBKLS0500) and ChemiDoc MP Imaging System (Bio-Rad) were used to image the blots. The blot densities were analyzed using Image Lab 6.0.1 software (Bio-Rad). Primary antibodies used in this study are listed in Table 2.

TABLE 2

| Antibodies | Catalog number | Dilution |
|---|---|---|
| CHAT | PA5-29653 | 1:200 (IF) |
| ISL1 | PA5-27789 | 1:5000 (WB); 1:200 (IF) |
| OLIG2 | sc-293163 | 1:250 (IF) |
| HB9 | DSHB 81.5C10 | 1:40 (IF) |
| TUJ1 | BioLegend 801202 | 1:500 (IF) |
| TDP-43 | 10782-2AP | 1:100 (IF) |
| k48 | Boston Biochem A-101 | 1:1000 (WB) |
| ATF4 | SC-200 | 1:400 (WB) |
| CHOP | CST2895 | 1:250 (WB) |
| p62 | M162-3 | 1:3000 (WB) |
| LC3 | PM036 | 1:3000 (WB) |
| p-TAU | ab92676 | 1:1500 (WB); 1:100 (IF) |
| TAU | ab80579 | 1:1000 (WB) |
| p53 | sc-126 | 1:200 (WB) |
| y-H2AX | ab26350 | 1:3000 (WB) |
| p97 | MA3-004 | 1:3000 (WB) |
| GAPDH | CST2118 | 1:5000 (WB) |
| Filamin 1 | sc-17749 | 1:1000 (WB) |
| MCM6 | sc-393618 | 1:500 (WB) |
| NES | MA1-110 | 1:1000 (WB) |
| HSP 47 | sc-5293 | 1:200 (WB) |
| pRB1 | sc-377528 | 1:200 (WB) |
| E2F1 | sc-251 | 1:200 (WB) |
| CCND1 | sc-8396 | 1:100 (WB) |
| DHFR | PA5-30992 | 1:1000 (WB) |

### MN Cell Survival Assay

To compare p97 WT and R155H/+ cells, MNs were thawed and plated on poly-lysine coated 96-well plates (Greiner, cat #655090) at a density of 1000 cells per 100 μL per well in MN maturation medium as described in the “motor neuron differentiation and maturation” section. Medium was changed with 100 μL of the same medium containing 15 μg/mL geltrex on day 1. 100 μL additional medium (excluding Geltrex) was added on day 4. Half of the medium (excluding the Brainfast supplement and Geltrex) was changed on day 7 and 11. 200 nM Ara-C was added to inhibit non-MN cells from proliferating. On day 14, the medium was replaced with N2 medium (DMEM/F12 and neurobasal medium, 1:1 v/v, 1% 100× N2). N2 medium was changed every six days. Cell viability was monitored every 3 or 4 days by staining with Calcein AM Viability Dye (Thermo, cat #65-0853-81), acquiring and analyzing images with ImageXpress Micro Confocal High-Content Imaging System (Molecular Devices).

For p97 inhibitor treatment, MNs were plated on poly-lysine coated 384—well plates (Greiner, cat #781946) at a density of 500 cells per 30 μL per well. The same medium was used for plating and maintaining cells. Cell viability was monitored at day 1 and day 14. On day 14, the medium was replaced with N₂ medium containing DMSO or 400 nM of p97 inhibitors, and cell viability was tested after 6 days of treatment.

### Electrophysiological Recordings

The recording electrode area containing 26,400 platinum microelectrodes on the MaxTwo HD-MEA 6-well plate (MaxWell Biosystems AG) was pre-coated with poly-lysine. 2×105 MN cells and 4×104 astrocytes (purchased from iCell) were plated per well on the poly-lysine coated area. Medium and culture methods are the same as those described in the “motor neuron differentiation and maturation” section. Spontaneous electrical activity and networks were analyzed using the MaxTwo microelectrode array system (MaxWell Biosystems AG).

### Proteomics

After culture, MN cells were scraped off the plates, washed with DPBS, and centrifuged at 300 g for 4 min to remove supernatant. Pellets were frozen at −80° C. The mass spec samples were prepared by following instructions for the EasyPep Mini MS Sample Prep Kit (Thermo Scientific, cat #A4006). Samples were dried using a vacuum centrifuge and resuspended in 0.1% formic acid (Thermo, cat #85178) water solution. Peptide concentrations were measured using the Quantitative Fluorometric Peptide Assay kit (Thermo, cat #23290).

LC-MS/MS experiments were performed by loading 0.5 μg sample onto an EASY-nLC 1000 (Thermo Scientific) connected to an Orbitrap Eclipse Tribrid mass spectrometer (Thermo Scientific). Peptides were separated on an Aurora UHPLC Column (25 cm×75 μm, 1.6 μm C₁₈, AUR2-25075C18A, Ion Opticks) with a flow rate of 0.4 μL/min and a total duration time of 131 min following the gradient composed of 3% Solvent B for 1 min, 3-19% B for 72 min, 19-29% B for 28 min, 29-41% B for 20 min, 41-95% B for 3 min, and 95-98% B for 7 min. Solvent A consists of 97.8% H₂O, 2% ACN, and 0.2% formic acid; solvent B consists of 19.8% H₂O, 80% ACN, and 0.2% formic acid. MS1 scans were acquired with a range of 400-1600 m/z in the Orbitrap at 120 k resolution. The maximum injection time was 50 ms, and the AGC target was 2×105. The filter dynamic exclusion was set to exclude after 1 time, 30 s duration, and 10 ppm mass tolerance. MS2 scans were acquired with quadrupole isolation mode and higher-energy collisional dissociation (HCD) activation type in the Iontrap. The isolation window was 1.4 m/z, collision energy was 35%, maximum injection time was 35 ms, and the AGC target was 1×104. Other global settings were as below: ion source type, NSI; spray voltage, positive ion 2400 V, negative ion 600 V; ion transfer tube temperature, 300° C. Method modifications and data collection were performed using Xcalibur software (Thermo Scientific).

Proteomic analysis was performed using Proteome Discoverer 2.4 (Thermo Scientific) software with the Uniprot human database and the SequestHT with Percolator validation. Protein abundance normalization was performed on total peptide. The data exported from PD2.4 were then used for further analysis. Limma analysis was performed using R studio following the user guide (Gordon, 2020). The volcano figures were plotted using Origin 2019b. PCA analyses were generated with PD2.4 and plotted using Prism 7. The Venn diagram was performed using FunRich 3.1. The heatmap figure was performed with prism 7. Functional enrichment analyses were performed with g:Profiler (Raudvere, 2019). The bubble plot was generated using Origin 2019b.

### Transcriptomics

RNA samples were prepared by following the same procedures described in the “Quantitative real-time PCR” method part. The integrity of RNA was assessed using RNA 6000 Pico Kit for Bioanalyzer (Agilent Technologies, cat #5067-1513), and mRNA was isolated using NEBNext Poly (A) mRNA Magnetic Isolation Module (NEB, cat #E7490). The Ultra II RNA Library Prep Kit for Illumina (NEB, cat #E7770) was used to construct RNA-seq libraries by following the manufacturer's instructions. Briefly (Gamez, 2020), mRNA isolated from ˜1 μg of total RNA was fragmented to the average size of 200 nt by incubating at 94° C. for 15 min in first strand buffer, cDNA was synthesized using random primers and ProtoScript II Reverse Transcriptase followed by second strand synthesis using NEB Second Strand Synthesis Enzyme Mix. The resulting DNA fragments were end-repaired, dA tailed and ligated to NEBNext hairpin adaptors (NEB, cat #E7335). After ligation, adaptors were converted to the ‘Y’ shape by treating with USER enzyme and DNA fragments were size selected using Agencourt AMPure XP beads (Beckman Coulter, cat #A63880) to generate fragment sizes between 250 and 350 bp. Adaptor-ligated DNA was PCR amplified, followed by AMPure XP bead clean up. Libraries were quantified with Qubit dsDNA HS kit (Thermo Scientific, cat #Q32854), and the size distribution was confirmed with the High Sensitivity DNA Kit for Bioanalyzer (Agilent Technologies, cat #5067-4626). Libraries were sequenced on Illumina HiSeq2500 in single read mode with a read length of 100 nt to the depth of 30 million reads per sample following manufacturer's instructions. Base calls were performed with RTA 1.13.48.0 followed by conversion to FASTQ with bcl2fastQ 1.8.4. The differential gene expression was performed with DESeq2 (Love, 2014).

### Example 1: Generation of iPSCs Carrying R155H p97 Mutation and Isogenic WT p97

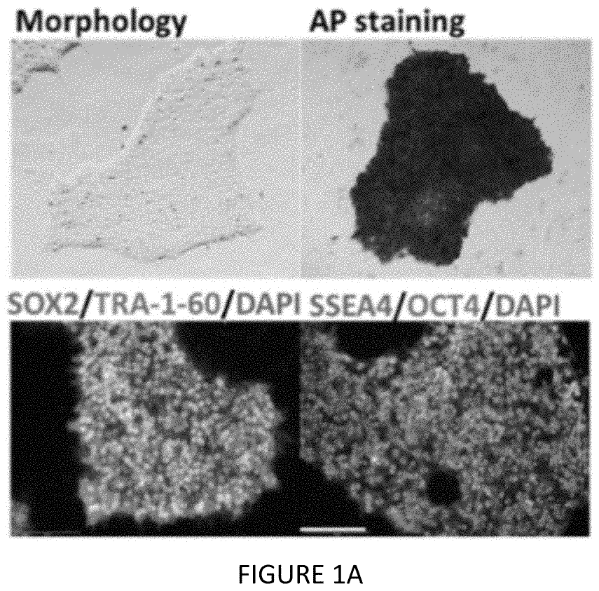

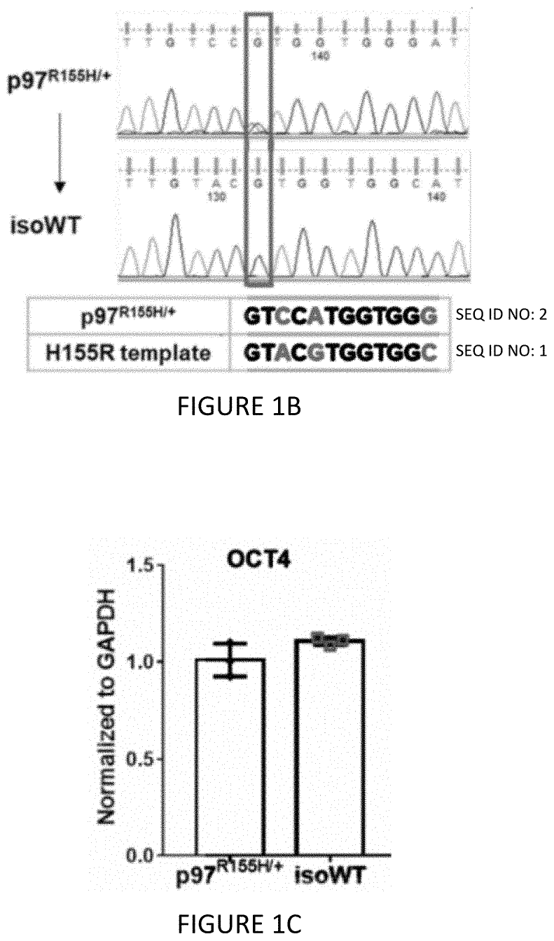

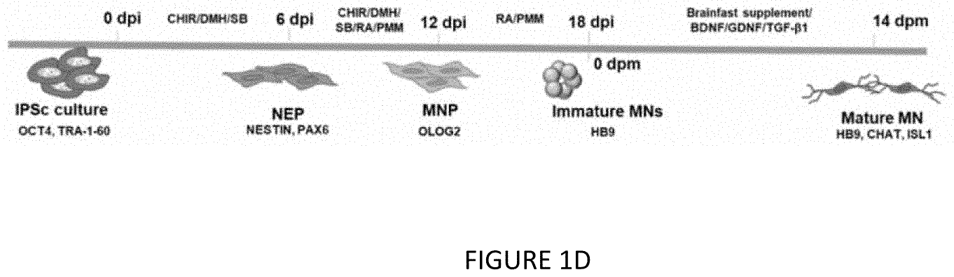

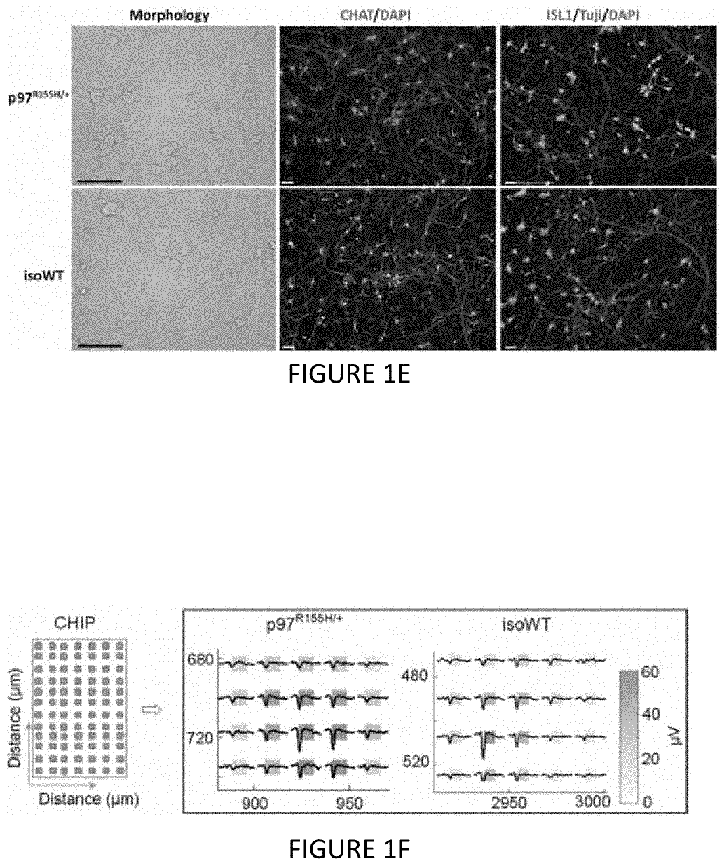

Neurons induced from reprogrammed patient cells is a powerful tool to explore mechanisms linked to CNS diseases, as well as to test potential therapies (Allsopp, 2019). To study the pathological effects of p97 mutation in human MNs, human iPSCs from IBMPFD patient fibroblasts harboring a heterozygous p97 mutation were generated (p97^{R155H/+}) through reprogramming (Takahashi, 2007). Cell morphology, alkaline phosphatase (AP) staining and immunofluorescence (IF) staining of four pluripotency markers (FIG. 1A) were used to characterize the iPSCs (Baghbaderani, 2016). It has been widely reported that genetic abnormalities can occur during iPSC generation and routine culture (Assou, 2018). To rule out abnormal iPSC clones, recurrent karyotypic abnormalities reported were tested in iPSCs using quantitative RT-PCR (qPCR) (Baker, 2016). Clones that passed this qPCR assay were sent for karyotyping after 10 passages, and the normal karyotype clones were used for differentiation. Comparing patient-derived cells with cells from genetically different healthy controls has been reported to overlook subtle phenotypes (Ben Jehuda, 2018). To eliminate the effects of genetic background, isogenic iPSCs (isoWT) were generated by correcting the p97 mutation (R155H) in patient iPSCs using CRISPR (Ben Jehuda, 2018). Isogenic iPSCs were confirmed using DNA sequencing (FIG. 1B). OCT4 RNA levels were the same in p97^{R155H/+} and isoWT iPSCs, indicating that the stem cell pluripotency of isoWT iPSCs was not affected by CRISPR (FIG. 1C).

### Example 2: Differentiation of iPSCs into Motor Neurons

One patient's iPSC line and its isogenic WT line were differentiated to MNs in triplicates using a published 18-day protocol (Du, 2015) (FIG. 1D). The expression of specific markers for each differentiation stage was detected using qPCR and IF staining. Following differentiation, the expression of OCT4, which maintains and supports induction of stem cell pluripotency (Shi, 2010) was significantly decreased. The expression of the neuroectodermal stem cell marker (Bernal, 2018), Nestin (NES), contentiously increased as cells progressed from neuroepithelial progenitors (NEPs) to motor neuron progenitors (MNPs). The highest expression of OLIG2 was observed in MNPs, and HB9 expression was observed in immature MNs at 18 days post induction (dpi). Consistent with our qPCR results, immunofluorescence signals indicated the presence of specific cell types: NES, SOX1 in NEPs, Oligo 2 in NMPs, and HB9 in immature MNs. To generate mature MNs, the immature MNs were re-plated and cultured for another 14 days using maturation media (FIG. 1D). At 14 days post maturation (dpm), we observed uniform neuron-like morphology in both p97^{R155H/+} and isoWT MNs (FIG. 1E). IF staining showed that both p97^{R155H/+} and isoWT cultures yielded >90% CHAT and ISL1 expressing MNs (FIG. 1E). In addition, spontaneous firing was observed from both p97^{R155H/+} and isoWT MNs using the Maxwell activity scan assay (FIG. 1F).

### Example 3: p97^{R155H/+} MNs Recapitulate Neurodegeneration

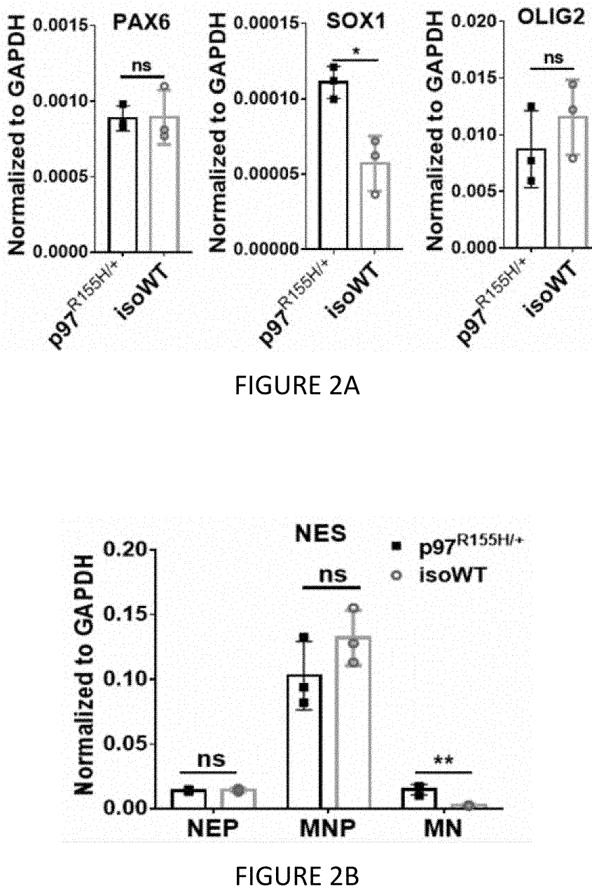

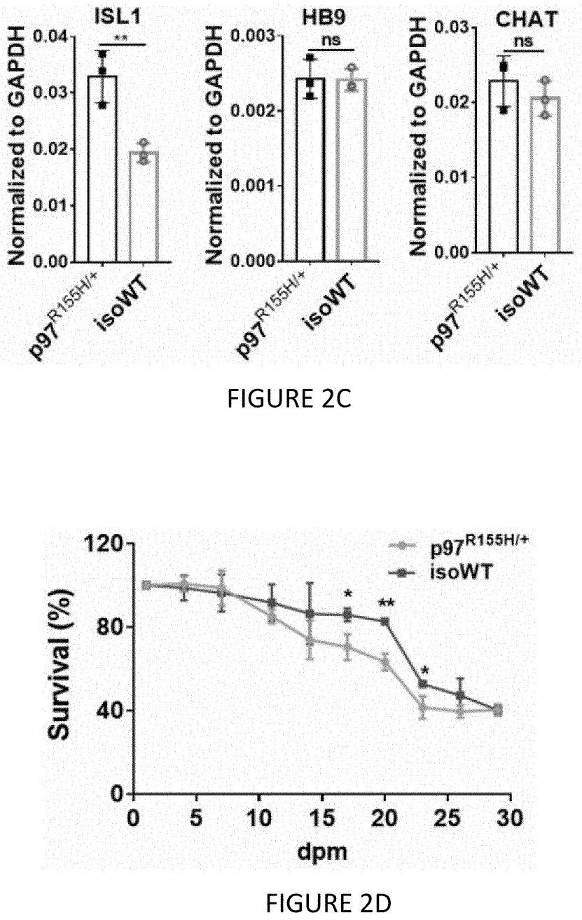

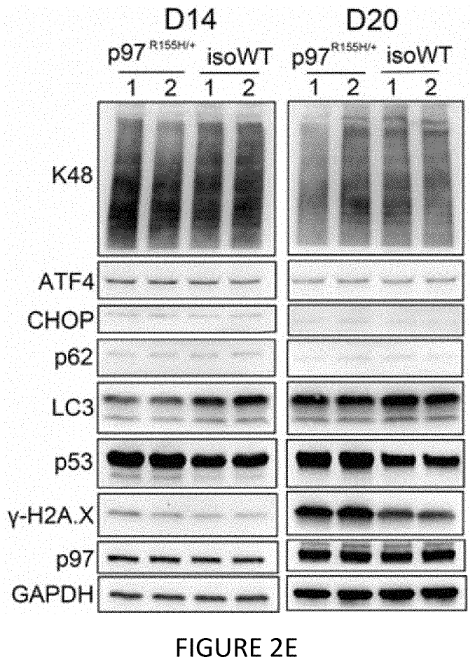

To assess the influence of p97^{R155H/+} on MN differentiation, the expression of PAX6 and SOX1 in NEPs, Olig2 in NMPs, ISL1, CHAT, HB9 in mature MNs and NES at all the 3 stages were compared between p97^{R155H/+} and isoWT. During differentiation, the increased expression of SOX1 was observed in p97^{R155H/+} NEps (FIG. 2A-B). Expression of the MN-specific marker, HB9, and mature MN marker, CHAT (Du, 2015), showed no difference between p97^{R155H/+} and isoWT motor neurons. However, the expression of NES (a stem cell marker) and ISL1 (the earliest marker of developing cholinergic neurons) (Allaway, 2017; Elshatory, 2008; Cho, 2014) were higher in p97^{R155H/+} MNs than in isoWT MNs at 14 dpm (FIG. 2B-FIG. 2C).

Neuron loss and death is a pathological feature of neurodegeneration (Gitcho, 2009). To check whether the induced p97^{R155H/+} MNs recapitulate this neurodegeneration, a cell survival assay was performed during MN maturation culture using Calcein-AM live cell staining (FIG. 2D). p97^{R155H/+} and isoWT MNs displayed no significant difference in cell survival from 1 to 14 dpm. However, between 17 and 23 dpm, significantly decreased neuron survival was observed in p97^{R155H/+} cultures (FIG. 2D).

To investigate the cellular effects of p97^{R155H/+} the MNs at 14 and 20 dpm, hereafter referred to as D14 and D20 MNs were harvested. Markers related to proteasomal degradation, autophagy, apoptosis and tauopathy were examined. The levels of p97, p62, proteasomal substrates (K48 poly-ubiquitinated substrates) and unfolded protein response (UPR) proteins (ATF4 and CHOP) were not affected by p97^{R155H/+} at both 14 and 20 dpm. Relative to controls, p97^{R155H/+} MNs showed decreased LC3 levels and increases in p53 and y-H2Ax from 14 to 20 dpm. The levels of Tau and phosphorylated Tau (p-Tau) showed no difference between p97^{R155H/+} and isoWT MNs at 14 dpm but showed a significant increase in p97^{R155H/+} MNs at 20 dpm. Consistent with western blot results, an increase in pTau staining was seen in p97^{R155H/+} MNs at 20 dpm. TDP43 staining displayed no difference between p97^{R155H/+} and isoWT MNs at 20 dpm. Taken together, markers of neurodegeneration are present in mature MNs at both D14 and D20.

### Example 4: Proteomic and Transcriptomic Profiling of Mature MNs

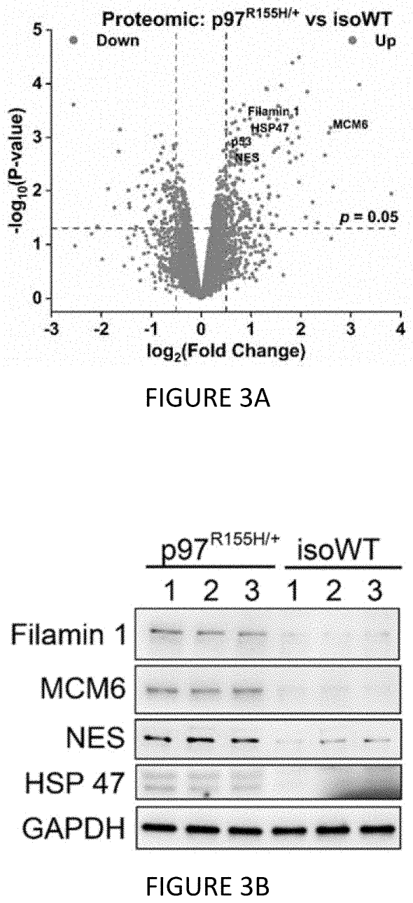

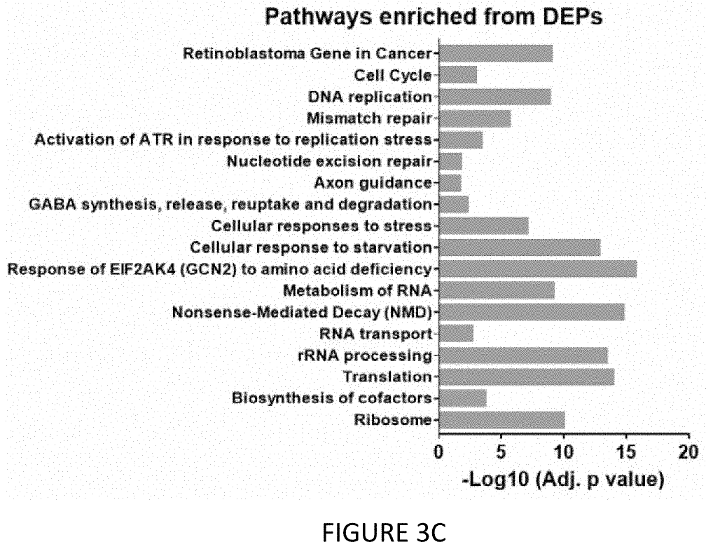

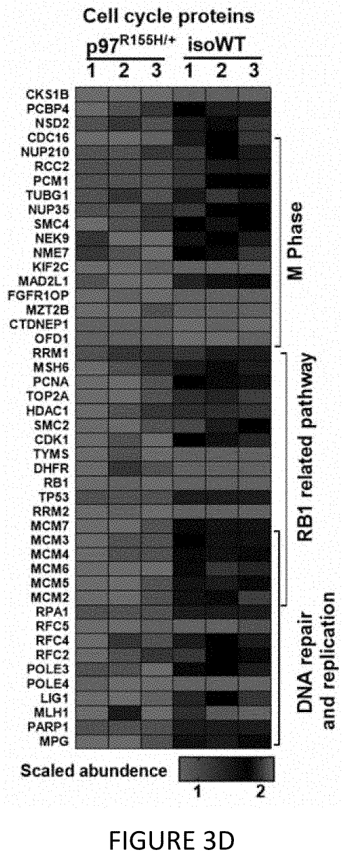

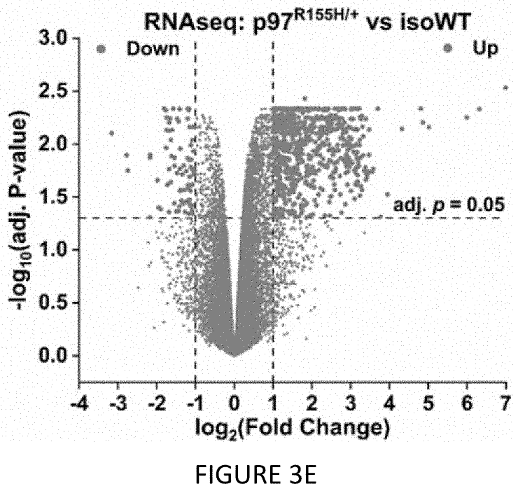

In order to elucidate the molecular mechanisms linked to p97^{R155H/+} driven neurodegeneration, a proteomic and transcriptomic analysis of D14 MNs were conducted to capture dysregulated markers at early time points in maturation. Three independent biological replicates were performed for both p97^{R155H/+} and isoWT MNs. The proteomic analysis was performed using label-free quantification. A total of 7101 proteins were identified (67131 peptides, FDR <1%) and quantified from all six samples. After excluding data with more than 1 missing value in both groups, a differential expression analysis was performed on the remaining 6043 proteins using Limma (Ritchie, 2015). 778 differentially expressed proteins (DEPs, p<0.05) (FIG. 3A) were identified. Of them, ISL1, NES and p53 were increased in p97^{R155H/+} MNs. These data are consistent with the qPCR and western blot results above (FIG. 2B-FIG. 2E). To further validate our proteomic data, the protein levels of Nestin, Filamin 1, MCM6 and HSP47 were determined by western blotting (FIG. 3B). Consistent with the proteomic data, all four proteins were increased in p97^{R155H/+} MNs. Functional enrichment analysis revealed 93 and 103 of the 778 DEPs are synapse and mitochondrion components respectively, which indicates that synapse and mitochondrion are potentially disrupted in p97-mutant MNs, as described previously (Hall, 2017). Cellular pathways related to translation, RNA metabolism, cellular response to stress, DNA repair and replication and cell cycle were also altered in p97^{R155H/+} MNs (FIG. 3C). In addition, many DEPs involved in DNA repair and replication and retinoblastoma protein (RB1) related pathways overlapped with cell cycle related DEPs. The majority of those DEPs were increased in p97^{R155H/+} MNs (FIG. 3D).

Using RNA-seq 706 differentially expressed genes (DEGs) were identified (Adj. p<0.05 and 1 log 2 (Fold Change)1>1) from 20629 genes. Of the 706 DEGs, 649 were upregulated and 57 were downregulated in p97^{R155H/+} MNs (FIG. 3E). Consistent with the proteomic analysis, functional enrichment analysis on DEGs revealed that cell cycle, DNA replication and RB1 related pathways were potentially dysregulated in p97^{R155H/+} MNs (FIG. 3E). Of the DEGs, all 113 cell cycle related genes were upregulated in p97^{R155H/+} MNs. Compared to the proteomics analysis, 35 of the 46 cell cycle related DEPs, were upregulated on both the protein and RNA level (FIG. 3E).

### Example 5: p97^{R155H/+} Upregulates the RB1/E2F1 Pathway in Mature MNs

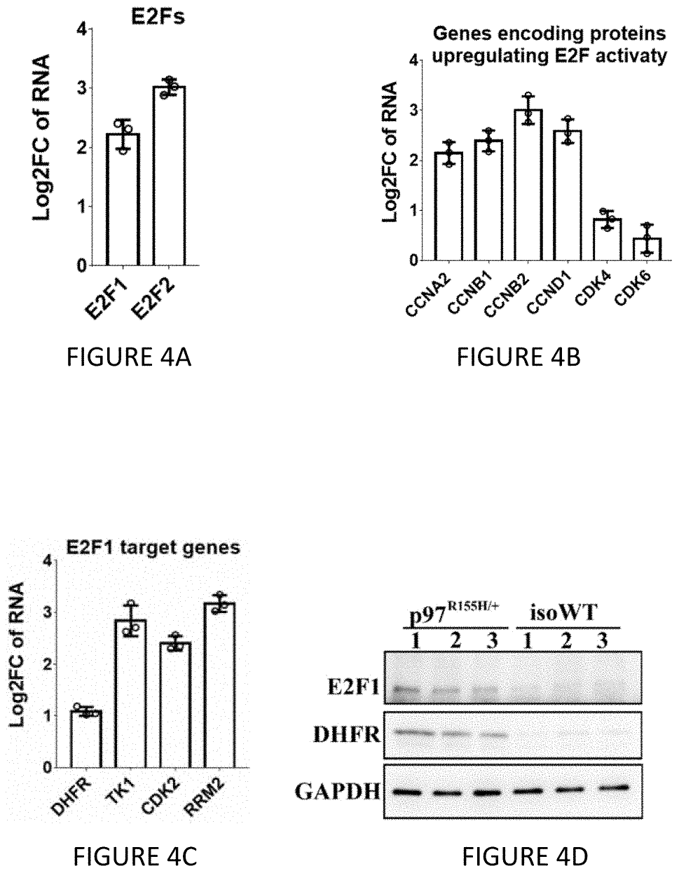

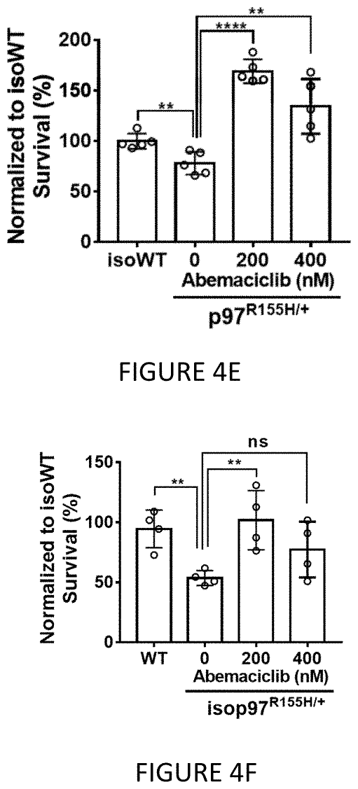

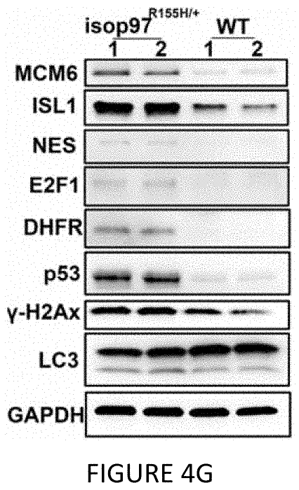

Both the proteomic and RNA-seq data revealed increased expression of cell cycle related genes and proteins in p97^{R155H/+} motor neurons. This indicates that the cell cycle is potentially deregulated in mature p97^{R155H/+} MNs. The RB1/E2F pathways play important roles in cell cycle control as genes encoding DNA replication and cell cycle regulatory factors are regulated by E2Fs (Ishida, 2001). The overexpression of E2F1 in quiescent cells leads to the induction of cellular DNA synthesis and apoptosis (Johnson, 1993, Kowalik, 1995). RNA-seq data indicates that E2F1 and E2F2 were upregulated in p97^{R155H/+} MNs (FIG. 4A). Genes encoding proteins which positively regulate E2F pathways, including CCND1 and CDK4/6, were also upregulated in p97^{R155H/+} MNs (FIG. 4B). The upregulation of E2F1, CDKs and cyclins suggests that E2F1 transcriptional activity is increased. Indeed, known E2F1 target genes, including DHFR, CDK2, RRM2 and TK1 (Wells, 2002; Chen, 2012; Fang, 2015) are upregulated (FIG. 4C). In addition, CDKs and cyclins inactivate RB1 by phosphorylation and release E2F1 to induce the transcription of cell cycle genes (Indovina, 2015). Levels of phosphorylated RB1 (pRB1) were increased in p97^{R155H/+} MNs (FIG. 4D). Moreover, the protein levels of CCND1, E2F1 and DHFR were significant elevated in p97^{R155H/+} MNs (FIG. 4D). These data suggest that the RB1/E2F1 pathway is upregulated in p97^{R155H/+} MNs.

Cell cycle deregulation has been observed in multiple NDs, including AD, ALS and SMA. RB1/E2F1 pathway is also linked to cell fate decisions and the induction of apoptosis. To evaluate whether the upregulation of E2F1 pathway is associated with the cell death of p97^{R155H/+} MNs, In particular, RB1/E2F1 pathway was inhibited through treatments with an FDA-approved CDK4/6 inhibitor, Abemaciclib. p97^{R155H/+} and iso p97^{R155H/+} MNs were treated with DMSO, 200 nM or 400 nM Abemaciclib from 8 dpm, and viable cells were determined by live cell staining every 6 days. Both 200 nM and 400 nM abemaciclib treatments improved the viability of p97^{R115H/+} MNs and iso p97^{R155H/+} MNs at 26 dpm (FIG. 4E and FIG. 4F). These data suggest that the upregulation of RB1/E2F1 pathway may be related to the cell death of p97^{R155H/+} MNs and CDK4/6 inhibitor can be used to promote MN survival.

To determine whether the dysregulated proteins observed were due to the specific genetic background of this particular patient or p97^{R155H/+} mutant, another isogenic pair of MNs derived from an unaffected individual carrying WT p97 and differentiated into MNs were generated using the same method as described above (FIG. 1D) and harvested at 14 dpm. As shown in FIG. 4G, the upregulation of cell cycle protein (MCM6), cell death associated proteins (p53, γ-H2Ax), E2F1 pathway (E2F1, DHFR), NES, ISL1, and the dysregulation of LC3 were also observed in the iso p97^{R155H/+} MNs. These results are consistent with p97^{R155H/+} and isoWT MNs and indicate that the dysregulation of those proteins was caused by by p97^{R155H/+} mutant.

### Example 6: p97 Inhibitors Relieve p97^{R155H/+}-Driven Neurodegeneration

Cell cycle deregulation has been observed in multiple NDs, including AD, ALS and SMA (Joseph, 2020; Hor, 2018). RB1/E2F1 pathway is also linked to cell fate decisions and the induction of apoptosis (Indovina, 2015). p97 has been implicated in cell cycle regulation and DNA replication (Zhang, 1999; Mouysset, 2008), inhibition of p97 downregulates CCND1 (Parisi, 2018) and blocks p97 disease mutant phenotypes in Drosophila (Zhang, 2017). To test whether two potent p97 inhibitors rescue p97^{R155H/+} MNs from neurodegeneration, the p97^{R155H/+} MNs were treated with CB-5083 and NMS-873 at 14 dpm. After 6 days of treatment, both CB-5083 and NMS-873 significantly reduced the loss of MNs (FIG. 5A and FIG. 5B). In addition, the treatments reversed increases in ISL1, NES, Tau, pTau, MCM6, p53, y-H2Ax and slightly elevated LC3 in p97^{R155H/+} MNs (FIG. 5C).

To reveal the global effects of p97 inhibitors on p97^{R155H/+} MNs, proteomic analysis was performed on the D20 MNs after 6 days of treatment with DMSO, CB-5083 or NMS-873. Principal-component analysis of proteomic data revealed a clear separation between isoWT and p97^{R155H/+} MNs along principal component 1 (PC1). Treatment with p97 inhibitors reduced the distance between isoWT and p97^{R155H/+} MNs along PC1 (FIG. 5D). 1360 DEPs (p<0.05) in D20 MNs treated with DMSO were identified. Consistent with the western blot results, proteomics analysis revealed that both CB-5083 and NMS-873 reduced Tau and MCM6 in p97^{R155H/+} MNs. 428 of the 920 upregulated proteins were reduced and 192 of the 440 downregulated proteins were increased after the treatment with CB-5083 or NMS-873 (FIG. 5E). To further clarify the cellular effects of p97 inhibitors, the most significantly affected DEPs were listed, with 1 log 2 (Fold Change) 1>1, which were corrected to WT level by CB-5083 or NMS-873 (FIG. 5F). Functional enrichment analysis revealed that deregulation of cellular pathways in D20 p97^{R155H/+} MNs are involved in DNA repair and replication, and metabolism of RNA related pathways (FIG. 5G, lane 1). Surprisingly, cell cycle related pathways were enriched only at D14 (FIG. 3A-3E) but not at D20. Of the 46 dysregulated cell cycle proteins identified from D14 MNs, only 16 exhibited increases in D20 p97^{R155H/+} MNs. The fold change in those 16 proteins in D20 MNs is lower than that in D14 MNs. The dysregulated proteins which were reversed by p97 inhibitors are linked to RNA metabolism associated pathways (FIG. 5G, lane 2), and the DEPs which were not corrected by p97 inhibitors are linked to DNA replication (FIG. 5G, lane 3).

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

### REFERENCES

Allaway, K. C. and R. Machold, Developmental specification of forebrain cholinergic neurons. Dev Biol, 2017. 421(1): p. 1-7.
Allsopp, T. E., A. Ebneth, and A. Cabrera-Socorro, Deploying human pluripotent stem cells to treat central nervous system disorders: facts, challenges and realising the potential. Stem Cell Res, 2019. 41: p. 101581.
Al-Obeidi, E., et al., Genotype-phenotype study in patients with valosin-containing protein mutations associated with multisystem proteinopathy. Clin Genet, 2018. 93(1): p. 119-125.
Assou, S., J. Bouckenheimer, and J. De Vos, Concise Review: Assessing the Genome Integrity of Human Induced Pluripotent Stem Cells: What Quality Control Metrics? Stem Cells, 2018. 36(6): p. 814-821.
Badadani, M., et al., VCP associated inclusion body myopathy and Paget disease of bone knock-in mouse model exhibits tissue pathology typical of human disease. PLoS One, 2010. 5(10).
Baghbaderani, B. A., et al., Detailed Characterization of Human Induced Pluripotent Stem Cells Manufactured for Therapeutic Applications. Stem Cell Rev Rep, 2016. 12(4): p. 394-420.
Baker, D., et al., Detecting Genetic Mosaicism in Cultures of Human Pluripotent Stem Cells. Stem Cell Reports, 2016. 7(5): p. 998-1012.
Ben Jehuda, R., Y. Shemer, and O. Binah, Genome Editing in Induced Pluripotent Stem Cells using CRISPR/Cas9. Stem Cell Rev Rep, 2018. 14(3): p. 323-336.
Bernal, A. and L. Arranz, Nestin-expressing progenitor cells: function, identity and therapeutic implications. Cell Mol Life Sci, 2018. 75(12): p. 2177-2195.
Blythe, E. E., et al., Ubiquitin- and ATP-dependent unfoldase activity of P97/VCP*NPLOC4*UFD1L is enhanced by a mutation that causes multisystem proteinopathy. Proc Natl Acad Sci USA, 2017. 114(22): p. E4380-E4388.
Burkhardt, M. F., et al., A cellular model for sporadic ALS using patient-derived induced pluripotent stem cells. Mol Cell Neurosci, 2013. 56: p. 355-64.
Chen, T., et al., The retinoblastoma protein selectively represses E2F1 targets via a TAAC DNA element during cellular senescence. J Biol Chem, 2012. 287(44): p. 37540-51.
Cho, H. H., et al., Isl1 directly controls a cholinergic neuronal identity in the developing forebrain and spinal cord by forming cell type-specific complexes. PLoS Genet, 2014. 10(4): p. e1004280.
Chou, T. F. and R. J. Deshaies, Development of p97 AAA ATPase inhibitors. Autophagy, 2011. 7(9): p. 1091-2.
Custer, S. K., et al., Transgenic mice expressing mutant forms VCP/p97 recapitulate the full spectrum of IBMPFD including degeneration in muscle, brain and bone. Hum Mol Genet, 2010. 19(9): p. 1741-55.
Darwich, N. F., et al., Autosomal dominant VCP hypomorph mutation impairs disaggregation of PHF-tau. Science, 2020. 370(6519).
Du, Z. W., et al., Generation and expansion of highly pure motor neuron progenitors from human pluripotent stem cells. Nat Commun, 2015. 6: p. 6626.
Elshatory, Y. and L. Gan, The LIM-homeobox gene Islet-1 is required for the development of restricted forebrain cholinergic neurons. J Neurosci, 2008. 28(13): p. 3291-7.
Fang, Z., et al., E2F1 promote the aggressiveness of human colorectal cancer by activating the ribonucleotide reductase small subunit M2. Biochem Biophys Res Commun, 2015. 464(2): p. 407-15.
Fernandez-Saiz, V. and A. Buchberger, Imbalances in p97 co-factor interactions in human proteinopathy. EMBO Rep, 2010. 11(6): p. 479-85.
Forman, M. S., et al., Novel ubiquitin neuropathology in frontotemporal dementia with valosin-containing protein gene mutations. J Neuropathol Exp Neurol, 2006. 65(6): p. 571-81.
Fujimori, K., et al., Modeling sporadic ALS in iPSC-derived motor neurons identifies a potential therapeutic agent. Nat Med, 2018. 24(10): p. 1579-1589.
Gamez, S., et al., The Developmental Transcriptome of Aedes albopictus, a Major Worldwide Human Disease Vector. G3 {Bethesda), 2020. 10{3): p. 1051-1062.
Gitcho, M. A., et al., VCP mutations causing frontotemporal lobar degeneration disrupt localization of TDP-43 and induce cell death. J Biol Chem, 2009. 284(18): p. 12384-98.
Gordon K. Smyth, M. R., Natalie Thorne, et al., limma: Linear Models for Microarray and RNA-Seq Data User's Guide. 2020.
Hall, C. E., et al., Progressive Motor Neuron Pathology and the Role of Astrocytes in a Human Stem Cell Model of VCP-Related ALS. Cell Rep, 2017. 19(9): p. 1739-1749.
Hor, J. H., et al., Cell cycle inhibitors protect motor neurons in an organoid model of Spinal Muscular Atrophy. Cell Death Dis, 2018. 9(11): p. 1100.
Indovina, P., et al., RB1 dual role in proliferation and apoptosis: cell fate control and implications for cancer therapy. Oncotarget, 2015. 6(20): p. 17873-90.
Ishida, S., et al., Role for E2F in control of both DNA replication and mitotic functions as revealed from DNA microarray analysis. Mol Cell Biol, 2001. 21(14): p. 4684-99.
Johnson, D. G., et al., Expression of transcription factor E2F1 induces quiescent cells to enter S phase. Nature, 1993. 365(6444): p. 349-52.
Johnson, J. O., et al., Exome sequencing reveals VCP mutations as a cause of familial ALS. Neuron, 2010. 68(5): p. 857-64.
Joseph, C., et al., Cell Cycle Deficits in Neurodegenerative Disorders: Uncovering Molecular Mechanisms to Drive Innovative Therapeutic Development. Aging Dis, 2020. 11(4): p. 946-966.
Kimonis, V. E., et al., Clinical studies in familial VCP myopathy associated with Paget disease of bone and frontotemporal dementia. Am J Med Genet A, 2008. 146A(6): p. 745-57.
Kimonis, V. E., et al., VCP disease associated with myopathy, Paget disease of bone and frontotemporal dementia: review of a unique disorder. Biochim Biophys Acta, 2008. 1782(12): p. 744-8.
Kimonis, V., Inclusion Body Myopathy with Paget Disease of Bone and/or Frontotemporal Dementia. GeneReviews, 2019.
Kowalik, T. F., et al., E2F1 overexpression in quiescent fibroblasts leads to induction of cellular DNA synthesis and apoptosis. J Virol, 1995. 69(4): p. 2491-500.
Love, M. I., W. Huber, and S. Anders, Moderated estimation of fold change and dispersion for RNA-seq data with DESeq2. Genome Biol, 2014. 15{12): p. 550.
Ludtmann, M. H. R., et al., Mutations in valosin-containing protein (VCP) decrease ADP/ATP translocation across the mitochondrial membrane and impair energy metabolism in human neurons. J Biol Chem, 2017. 292(21): p. 8907-8917.
Marchetto, M. C., et al., Induced pluripotent stem cells (iPSCs) and neurological disease modeling: progress and promises. Hum Mol Genet, 2011. 20(R2): p. R109-15.
Martin, L. J., et al., Mechanisms for neuronal degeneration in amyotrophic lateral sclerosis and in models of motor neuron death (Review). Int J Mol Med, 2000. 5(1): p. 3-13.
Mehta, S. G., et al., Genotype-phenotype studies of VCP-associated inclusion body myopathy with Paget disease of bone and/or frontotemporal dementia. Clin Genet, 2013. 83(5): p. 422-31.
Mouysset, J., et al., Cell cycle progression requires the CDC-48UFD-1NPL-4 complex for efficient DNA replication. Proc Natl Acad Sci USA, 2008. 105(35): p. 12879-84.
Nalbandian, A., et al., The homozygote VCP(R(1)(5)(5)H/R(1)(5)(5)H) mouse model exhibits accelerated human VCP-associated disease pathology. PLoS One, 2012. 7(9): p. e46308.
Neumann, M., et al., TDP-43 in the ubiquitin pathology of frontotemporal dementia with VCP gene mutations. J Neuropathol Exp Neurol, 2007. 66(2): p. 152-7.
Okita, K., et al., An efficient nonviral method to generate integration-free human-induced pluripotent stem cells from cord blood and peripheral blood cells. Stem Cells, 2013. 31{3): p. 458-66.
Parisi, E., et al., Cdc48/p97 segregase is modulated by cyclin-dependent kinase to determine cyclin fate during G1 progression. EMBO J, 2018. 37(16).
Penney, J., W. T. Ralvenius, and L. H. Tsai, Modeling Alzheimer's disease with iPSC-derived brain cells. Mol Psychiatry, 2020. 25(1): p. 148-167.
Ranganathan, S. and R. Bowser, Alterations in (11 to S Phase Cell-Cycle Regulators during Amyotrophic Lateral Sclerosis. The American Journal of Pathology, 2003. 162(3): p. 823-835.
Raudvere, U., et al., g:Profiler: a web server for functional enrichment analysis and conversions of gene lists {2019 update). Nucleic Acids Res, 2019. 47{W1): p. W191-W198.
Ritchie, M. E., et al., limma powers differential expression analyses for RNA-sequencing and microarray studies. Nucleic Acids Res, 2015. 43(7): p. e47.
Shi, G. and Y. Jin, Role of Oct4 in maintaining and regaining stem cell pluripotency. Stem Cell Res Ther, 2010. 1(5): p. 39.
Sison, S. L., et al., Using Patient-Derived Induced Pluripotent Stem Cells to Identify Parkinson's Disease-Relevant Phenotypes. Curr Neurol Neurosci Rep, 2018. 18(12): p. 84.
Takahashi, K., et al., Induction of Pluripotent Stem Cells from Adult Human Fibroblasts by Defined Factors. Cell, 2007. 131(5): p. 861-872.
Tang, W. K. and D. Xia, Mutations in the Human AAA(+) Chaperone p97 and Related Diseases. Front Mol Biosci, 2016. 3: p. 79.
Weihl, C. C., A. Pestronk, and V. E. Kimonis, Valosin-containing protein disease: inclusion body myopathy with Paget's disease of the bone and fronto-temporal dementia. Neuromuscul Disord, 2009. 19(5): p. 308-15.
Wells, J., et al., The identification of E2F1-specific target genes. Proc Natl Acad Sci US A, 2002. 99(6): p. 3890-5.
Xia, D., W. K. Tang, and Y. Ye, Structure and function of the AAA+ ATPase p97/Cdc48p. Gene, 2016. 583(1): p. 64-77.
Yi, L., et al., Altered intracellular localization and valosin-containing protein (p97 VCP) interaction underlie ATP7A-related distal motor neuropathy. Hum Mol Genet, 2012. 21(8): p. 1794-807.
Zhang, S. H., et al., Identification of the cell cycle regulator VCP (p97/CDC48) as a substrate of the band 4.1-related protein-tyrosine phosphatase PTPH1. J Biol Chem, 1999. 274(25): p. 17806-12.
Zhang, T., et al., Valosin-containing protein (VCP/p97) inhibitors relieve Mitofusin-dependent mitochondrial defects due to VCP disease mutants. Elife, 2017. 6.

Zhang, X., et al., Altered cofactor regulation with disease-associated p97/VCP mutations. Proc Natl Acad Sci USA, 2015. 112(14): p. E1705-14.

What is claimed is:

1. A method of improving, ameliorating, or treating a motor neuron disease, the method comprising:
identifying a subject having a motor neuron disease, or a symptom thereof; and
administering an effective amount of an agent that promotes inhibition of p97 in the subject,
wherein the motor neuron disease or a symptom thereof is reduced after the administering.

2. The method of claim 1, wherein the motor neuron disease is amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, or post-polio syndrome.

3. The method of claim 1, wherein the agent that promotes inhibition of p97 is an inhibitory nucleic acid molecule, p97 binding antagonist, a genetic tool, and/or a small molecule inhibitor.

4. The method of claim 1, wherein the motor neuron disease is caused by at least one mutation in p97.

5. The method of claim 4, wherein the mutation in p97 is R155H, D395G, R191Q, or R155C.

6. The method of claim 1, wherein the subject having a motor neuron disease expresses one or more genes involved in the RB1/E2F1 pathway differently than in normal subjects.

7. The method of claim 6, wherein the genes involved in the RB1/E2F1 pathway comprise E2F1, E2F2, CCNDJ, CDK4, CDK6, DHFR, CDK2, pRB1, RRM2 or TK1.

8. The method of claim 3, wherein the inhibitory nucleic acid molecule is an antisense nucleic acid.

9. The method of claim 3, wherein the inhibitory nucleic acid molecule is a siRNA.

10. The method of claim 3, wherein the inhibitory nucleic acid molecule is a shRNA.

11. The method of claim 3, wherein the inhibitory nucleic acid molecule corresponds to or is complementary to at least a fragment of nucleic acid encoding p97.

12. The method of claim 3, wherein the p97 binding antagonist inhibits the binding of p97 to its binding partners.

13. The method of claim 12, wherein the p97 binding antagonist is an antibody against p97 or a fragment of p97.

14. The method of claim 13, wherein the antibody is a monoclonal, polyclonal or an antibody fragment selected from the group consisting of Fab, Fab′-SH, Fv, scFv, and (Fab′)₂ fragments.

15. The method of claim 3, wherein the genetic tool is selected from the group consisting of a CRISPR/Cas9 system, a zinc finger nuclease system, a TALEN system, a homing endonucleases system or a meganuclease system.

16. The method of claim 3, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.

17. A method of identifying a subject having a motor neuron disease, the method comprising detecting at least one of:
(a) a presence, a genetic change and/or level of p97;
(b) a level of a product of a gene of the subject selected from the group consisting of: Sox1 (or an ortholog thereof), NES (or an ortholog thereof), Is11 (or an ortholog thereof), p53 (or an ortholog thereof), γ-H2AX (or an ortholog thereof), Tau (or an ortholog thereof), p-Tau (or an ortholog thereof), Mcm6 (or an ortholog thereof), LC3 (or an ortholog thereof), or a combination of two or more of the listed genes.

18. The method of claim 17, wherein detecting a presence, a genetic change and/or a level of (a) and/or (b), wherein (a) and/or (b) are expressed differently and/or have a different genetic status in normal and subjects with motor neuron disease.

19. A method of improving, ameliorating, or treating a motor neuron disease, the method comprising:
detecting the genetic status, level, and/or expression of p97 in a subject;
comparing the genetic status, level, and/or expression of p97 in the subject to the genetic status, level and/or expression of p97 in the normal subject, wherein detection of an abnormal genetic status and/or a high level and/or expression of mutant p97 in the subject relative to the normal subject indicates the presence of a motor neuron disease in the subject; and
administering to the subject an effective amount of an agent that promotes inhibition of p97 in the subject, wherein the agent that promotes inhibition of p97 is selected from the group consisting of an inhibitory nucleic acid molecule, p97 binding antagonist, a genetic tool, and/or a small molecule inhibitor;
wherein the motor neuron disease or a symptom thereof is reduced after the administering.

20. The method of claim 19, wherein the motor neuron disease is amyotrophic lateral sclerosis (ALS), inclusion body myopathy associated with Paget disease of bone and frontotemporal dementia (IBMPFD), progressive bulbar palsy, primary lateral sclerosis, progressive muscular atrophy, spinal muscular atrophy, Kennedy's disease, or post-polio syndrome.

21. The method of claim 19, wherein the agent that promotes inhibition of p97 is an inhibitory nucleic acid molecule, p97 binding antagonist, a genetic tool, and/or a small molecule inhibitor.

22. The method of claim 19, wherein the motor neuron disease is caused by at least one mutation in p97.

23. The method of claim 22, wherein the mutation in p97 is R155H, D395G, R191Q, or R155C.

24. The method of claim 19, wherein the subject having a motor neuron disease expresses one or more genes involved in the RB1/E2F1 pathway differently than in normal subjects.

25. The method of claim 24, wherein the genes involved in the RB1/E2F1 pathway comprise E2F1, E2F2, CCNDJ, CDK4, CDK6, DHFR, CDK2, pRB1, RRM2 or TK1.

26. The method of claim 21, wherein the inhibitory nucleic acid molecule is an antisense nucleic acid.

27. The method of claim 21, wherein the inhibitory nucleic acid molecule is a siRNA.

28. The method of claim 21, wherein the inhibitory nucleic acid molecule is a shRNA.

29. The method of claim 21, wherein the inhibitory nucleic acid molecule corresponds to or is complementary to at least a fragment of nucleic acid encoding p97.

30. The method of claim 21, wherein the p97 binding antagonist inhibits the binding of p97 to its binding partners.

31. The method of claim 30, wherein the p97 binding antagonist is an antibody against p97 or a fragment of p97.

32. The method of claim 31, wherein the antibody is a monoclonal, polyclonal or an antibody fragment selected from the group consisting of Fab, Fab′-SH, Fv, scFv, and (Fab′)₂ fragments.

33. The method of claim 21, wherein the genetic tool is selected from the group consisting of a CRISPR/Cas9 system, a zinc finger nuclease system, a TALEN system, a homing endonucleases system or a meganuclease system.

34. The method of claim 21, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.


SEQUENCE LISTING (6 sequences)

SEQ ID NO: 1 - DNA, 12 bp, Artificial Sequence
  H155R Template
gtacgtggtggc

SEQ ID NO: 2 - DNA, 12 bp, Artificial Sequence
  R155H Template
gtccatggtggg

SEQ ID NO: 3 - DNA, 20 bp, Artificial Sequence
  Guide RNA
ccacagcacgcatcccacca

SEQ ID NO: 4 - DNA, 80 bp, Artificial Sequence
  H155R Reverse Complement
atctgtttccaccactttgaactccacagcacgcatgccaccacgtacaagaaaaatgtctcctgcgagagcaaacagta

SEQ ID NO: 5 - DNA, 21 bp, Artificial Sequence
  VCP-Ex5 Forward Primer
tggagttggggagaggtaggg

SEQ ID NO: 6 - DNA, 26 bp, Artificial Sequence
  VCP-Ex5 Reverse Primer
aaaatcggatactggaatcagggaga